(19) United States
(12) Patent Application Publication
(10) Pub. No.: US 2024/0247258 A1
(43) Pub. Date: Jul. 25, 2024

(54) OLIGONUCLEOTIDE AND TARGET RNA SITE-SPECIFIC EDITING METHOD

(71) Applicant: FREST Inc., Tokyo (JP)

(72) Inventors: Masatora FUKUDA, Fukuoka-shi (JP); Kanako NOSE, Fukuoka-shi (JP); Yohei TOMITA, Fukuoka-shi (JP)

(21) Appl. No.: 17/905,881

(22) PCT Filed: Mar. 9, 2021
(86) PCT No.: PCT/JP2021/009323

§ 371 Date: Sep. 8, 2022

(30) Foreign Application Priority Data
Mar. 12, 2020 (JP) 2020-043253

(51) Int. Cl. C12N 15/113; C12N 9/78

(52) U.S. Cl. CPC C12N 15/113; C12N 9/78; C12N 2310/11; C12Y 305/04004

(57) ABSTRACT

A target-editing guide oligonucleotide that introduces site-specific editing to target RNA, including a first oligonucleotide that identifies the target RNA, a second oligonucleotide that is linked to the 3′-side of the first oligonucleotide, a third oligonucleotide which has a base sequence capable of forming a complementary strand together with the second oligonucleotide, and a first linking group that links the 5′ end of the first oligonucleotide and the 3′ end of the third oligonucleotide to one another. The first oligonucleotide includes a target-corresponding nucleotide residue that corresponds to an adenosine residue in the target RNA; a nucleotide chain including at least 10 and no more than 24 residues that is linked to the 5′ side of the target-corresponding nucleotide residue and has a base sequence which is complementary to the target RNA; and a nucleotide chain including at least 2 and no more than 7 residues that is linked to the 3′ side of the target-corresponding nucleotide residue and has a base sequence which is complementary to the target RNA.

### CROSS-REFERENCE TO RELATED APPLICATIONS

This application is the U.S. National Phase under 35 U.S.C. § 371 of International Application No. PCT/JP2021/009323, filed Mar. 9, 2021, designating the U.S., and published in Japanese as WO 2021/182474 on Sep. 16, 2021, which claims priority to Japanese Patent Application No. 2020-043253, filed Mar. 12, 2020, the entire contents of which are incorporated herein by reference.

### SEQUENCE LISTING STATEMENT

The present application contains a Sequence Listing, which is being submitted via EFS-Web on even date herewith. The Sequence Listing is submitted in a file entitled “2023-01-19 Sequence Listing—KITO073.001APC” which was created on Jan. 19, 2023, and is approximately 19,502 bytes in size. This Sequence Listing is hereby incorporated by reference.

### TECHNICAL FIELD

The present invention relates to methods for site-specific editing of oligonucleotides and target RNAs.

### BACKGROUND ART

With the development of genome editing technology, the method of controlling biological phenomena by modifying the genetic information which is the blueprint of living organisms, namely the DNA information in cells, is beginning to be used in the fields of medical and drug discovery as an approach to treat diseases. Since DNA is a permanent and unchanging molecule in the cells, the effect of DNA modification remains permanently in the subject cell or subject organism. On the other hand, RNA is a nucleic acid molecule in which DNA information is copied, and unlike DNA, it is a transient genetic information molecule in which synthesis and decomposition are repeated. Therefore, modification of RNA information can confer a temporary non-permanent genetic information modification effect in the target organism. In other words, although RNA modification technology is a genetic modification technology like DNA modification, its properties are significantly different.

As an RNA modification technique, for example, International Publication No. 2016/097212 describes oligonucleotide constructs for site-specific editing of nucleotides in a target RNA sequence, which include a target-directed moiety containing an antisense sequence complementary to a part of the target RNA, and a recruitment moiety that can attach to and recruit an RNA editing entity that can edit nucleotides which are present in the cells. Further, for example, International Publication No. 2017/010556 describes a method for introducing site-specific RNA mutations in which double-strand specific adenosine deaminase (ADAR) is allowed to act on a complex of a target RNA and a target editing guide RNA.

### SUMMARY OF THE INVENTION

### Problems to be Solved by the Invention

The oligonucleotide construct described in International Publication No. 2016/097212 has a stem-loop structure with a specific repeated sequence as a recruitment portion in addition to the targeting site. In addition, the target editing guide RNA described in WO 2017/010556 has an antisense region and an ADAR binding region having a stem-loop structure consisting of a specific sequence. An objective of the present invention is to provide a target editing guide oligonucleotide capable of inducing site-specific editing.

The specific means for solving the aforementioned problem is as follows, and the present invention encompasses the following aspects. The first aspect is an oligonucleotide that induces site-specific editing on a target RNA (hereinafter also referred to as “target editing guide oligonucleotide”), comprising, a first oligonucleotide that specifies a target RNA, a second oligonucleotide linked to the 3′ side of the first oligonucleotide, a third oligonucleotide having a base sequence capable of forming a complementary strand with the second oligonucleotide, and a first linkage group that links the 5′ end of the first oligonucleotide to the 3′ end of the third oligonucleotide. The first oligonucleotide consists of a target corresponding nucleotide residue that corresponds to the adenosine residue in the target RNA, a nucleotide chain of 10 or more residues and 70 or less residues linked to the 5′ side of the target-corresponding nucleotide residue and having a base sequence complementary to the target RNA, and a nucleotide chain of 2 or more residues and 7 or less residues linked to the 3′ side of the target corresponding nucleotide residue and having a base sequence complementary to the target RNA.

### Means for Solving the Problems

The target editing guide oligonucleotide may further comprise a second linkage group that links the 3′ end of the second oligonucleotide to the 5′ end of the third oligonucleotide. The second linkage group may comprise a nucleotide chain with the number of residues being 4 or more and 20 or less. The number of residues in the second oligonucleotide may be 2 or more and 30 or less. The first linkage group may comprise a nucleotide chain with the number of residues being 8 or more and 50 or less.

The second embodiment is a method for site-specific editing of a target RNA, comprising contacting the oligonucleotide with a target RNA in the presence of adenosine deaminase. A method for site-specific editing of a target RNA may be performed in eukaryotic cells. Alternatively, the method for site-specific editing of a target RNA may be performed in vivo or in vitro.

### Effect of the Invention

According to the present invention, it is possible to provide a target editing guide oligonucleotide capable of inducing site-specific editing.

### BRIEF DESCRIPTION OF THE DRAWINGS

FIG. 1 It is a figure showing the edit-inducing activity of a target-editing guide RNA.

FIG. 2 It is a figure showing the result of electrophoresis of a cyclic target-editing guide RNA.

FIG. 3 It is a figure showing the result of electrophoresis of a cyclic target-editing guide RNA.

FIG. 4 It is a figure showing the degradation resistance of cyclic target-editing guide RNA.

FIG. 5 It is a figure showing the interaction of cyclic target-editing guide RNA with the target RNA.

FIG. 6 It is a figure showing the editing inducing activity of cyclic target editing guide RNA.

FIG. 7 It is a figure showing the edit-inducing activity of the target editing guide RNA depending on the ADAR subtype.

FIG. 8 It is a figure showing the editing induction activity of the target editing guide RNA depending on the ADAR subtype.

FIG. 9 It is a result of electrophoresis showing that cyclic targeted editing guide RNAs are generated in cells.

FIG. 10 It is a figure showing the edit-inducing activity of cyclic targeted editing guide RNA in cells.

FIG. 11 It is a figure showing the edit-inducing activity of cyclic target-editing guide RNA in cells.

FIG. 12 It is a figure showing the change over time in the editing induction of cyclic target-editing guide RNA in cells.

FIG. 13 It is a figure showing changes over time in protein expression levels induced by editing of cyclic target-editing guide RNA in cells.

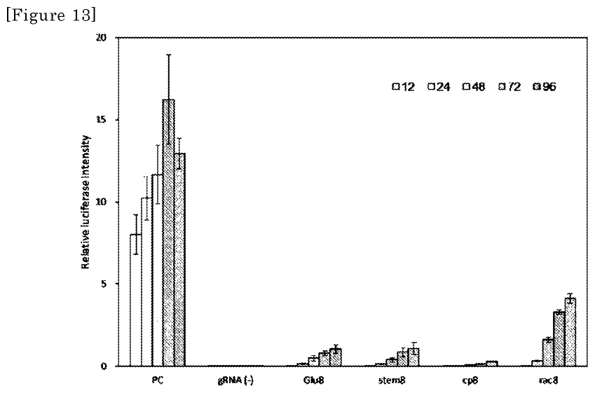

FIG. 14 It is a figure showing the editing induction activity of cyclic target editing guide RNAs by endogenous ADARs in cells.

FIG. 15 It is a figure showing the relationship between the length of the first linkage group and the editing-inducing activity of cyclic target-editing guide RNA.

### MODE FOR CARRYING OUT THE INVENTION

In the present specification, the term “process” is not only an independent process, but even if it cannot be clearly distinguished from other processes, it is included in this term as long as the intended purpose of the process is achieved. In addition, the content of each component in the composition means the total amount of the plurality of substances present in the composition when there are multiple substances corresponding to each component in the composition, unless otherwise specified. Furthermore, the upper and lower limits of the numerical ranges described herein can be arbitrarily selected and combined. Hereinafter, embodiments of the present invention will be described in detail. However, the embodiments shown below exemplify target editing guide oligonucleotides for embodying the technical idea of the present invention, and the present invention is not limited to the target editing guide oligonucleotides shown below.

### Target Editing Guide Oligonucleotide

The oligonucleotide that induces site-specific editing of the target RNA comprises a first oligonucleotide identifying the target RNA, a second oligonucleotide linked to the 3′ side of the first oligonucleotide, and a third oligonucleotide having a base sequence capable of forming a complementary strand with a second oligonucleotide, and a first linkage group connecting the 5′ end of the first oligonucleotide to the 3′ end of the third oligonucleotide, and induces site-specific editing of the target RNA. The first oligonucleotide consists of the target-corresponding nucleotide residue corresponding to an adenosine residue in the target RNA, an oligonucleotide of 10 or more and 24 or less residues linked to the 5′ side of the target-corresponding nucleotide residue and having a base sequence complementary to the target RNA, and an oligonucleotide of 2 or more and 7 or less residues linked to the 3′ side of the target-corresponding nucleotide residue and having a base sequence complementary to the target RNA.

In the target editing guide oligonucleotide, the first oligonucleotide is thought to function as a complementary region (antisense region; ASR) to the target RNA. In addition, the second oligonucleotide linked to the 3′ side of the first oligonucleotide forms a double strand with the third oligonucleotide and is thought to function as an editing enhancing region, an ADAR binding region (ADAR recruiting region; ARR), etc. Such constructs allow for site-specific editing of target RNAs by ADARs. It is thought that the third oligonucleotide is linked to the first oligonucleotide via the first linking group, thereby facilitating the formation of a duplex with the second oligonucleotide.

The target editing guide oligonucleotide induces site-specific editing to the target RNA, for example, by specifically recruiting an ADAR that catalyzes target editing to the target RNA. ADAR is an enzyme that converts adenosine residues in double-stranded RNA into inosine residues by a hydrolytic deamination reaction, and is widely present in mammalian cells. Since the inosine residue is similar in structure to the guanosine residue, it is translated as a guanosine residue at translation of the RNA information, and as a result, the RNA information is edited. When such RNA editing occurs in the portion encoding the amino acid, amino acid substitution or the like occurs even though there is no DNA mutation on the genome. ADARs that exist in the mammalian species include ADAR1, ADAR2, and ADAR3, which are different genes. Target-editing guide oligonucleotides enhance the target-editing activity of ADAR1 or ADAR2 among them. When introduced into mammalian cells, target-editing guide oligonucleotides recruit ADARs present in the cells to the target RNA and can induce site-specific editing of the target RNA.

The first oligonucleotide identifies a target RNA. The target RNA is not particularly limited as long as it comprises an adenosine residue to be the target of editing, and it may be either cellular RNA or viral RNA, and is usually pre-mRNA or mRNA encoding a protein. Editing sites in the target RNA may be present in untranslated regions, splice regions, exons, introns, or any region that affects RNA stability, structure or function. Further, the target RNA may also contain a mutation to be corrected or altered. Alternatively, the target RNA may be one whose sequence has been mutated to encode a different phenotype than the native form.

The target RNA is preferably an RNA that encodes a protein. Specific examples of the encoded protein include phosphorylated proteins involved in signal transduction such as serotonin receptors, glutamate receptors, voltage-gated potassium channels, STAT3, NFkBIA, and MAPK14.

Target editing guide oligonucleotides can be applied, for example, to the treatment of hereditary diseases. Hereditary diseases include cystic fibrosis, leukoderma, α-1 antitrypsin deficiency, Alzheimer's disease, muscular atrophic lateral sclerosis, asthma, ß-thalassemia, CADASIL syndrome, Charcot-Marie-Tooth disease, chronic obstructive lung disease (COPD), distal spinal muscle atrophy (DSMA), Duchenne/Becker muscular dystrophy, dystrophic Epidermolysis bullosa, Epidermylosis bullosa, Fabry disease, Factor V Leiden-related disorders, familial adenomas polyposis, galactosemia, Gaucher disease, glucose-6-phosphate dehydrogenase deficiency, hemophilia, hereditary hemochromatosis, Hunter syndrome, Huntington's disease, Hurler syndrome, inflammatory bowel disease (IBD), hereditary polyagglutination syndrome, Leber's congenital amaurosis, Lesch-Nyhan syndrome, Lynch syndrome, Marfan syndrome, Mucopolysaccharidosis, muscular dystrophy, Myotonic dystrophy types I and II, Neurofibromatosis, Niemann-Pick disease types A, B and C, NY-eso1-related pancreatic cancer, Parkinson's disease, Peutz-Jeghers syndrome, phenylketonuria, Pompe disease, Primary ciliary dyskinesia, prothrombin mutation-related diseases such as prothrombin G20210A mutation, pulmonary hypertension, retinitis pigmentosa, Sandhoff disease, severe combined immunodeficiency syndrome (SCID), sickle cell anemia, spinal muscle atrophy, Stargardt disease, Tay-Sachs disease, Usher syndrome, X-linked immunodeficiency, and various forms of cancer (for example, BRCA1 and 2 related breast cancer, ovarian cancer, etc.).

The first oligonucleotide consists of a target-corresponding nucleotide residue that corresponds to the adenosine residue in the target RNA, a 5′ side oligonucleotide chain of 10 or more residues and 70 residues or less linked to the 5′ side of the target-corresponding nucleotide residue and having a base sequence complementary to the target RNA, and a 3′ side oligonucleotide chain of 2 or more residues and 7 or less residues linked to the 3′ side of the target-corresponding nucleotide residue and having a base sequence complementary to the target RNA.

The oligonucleotide strands respectively linked to the 5′ and 3′ sides of the target corresponding nucleotide residue form a double strand with the target RNA to form a complementary strand as a whole, and the target RNA and the editing target site in the target RNA are thereby identified. Here, the complementary base sequences include, in addition to base sequences capable of forming Watson-Crick base pairs, thermodynamically stable non-Watson-Crick base pairs such as GU base pairs.

The target-corresponding nucleotide residue is a nucleotide residue that corresponds to an adenosine residue that is an editing target, such as a cytidine residue, a uridine residue, an adenosine residue, or a derivative thereof. The target-corresponding nucleotide residue is preferably a base that does not form a base pair with the adenosine residue that is the editing target, more preferably a cytidine residue or a derivative thereof, still more preferably a cytidine residue.

The nucleotide sequences of the oligonucleotide chains linked to the 5′ side and 3′ side of the target-corresponding nucleotide residue are respectively complementary nucleotide sequences to the corresponding nucleotide sequences of the target RNA. From the viewpoint of specificity to the target RNA, the number of residues of the 5′-side oligonucleotide chain linked to the 5′ side of the target-corresponding nucleotide residue is, for example, 10 or more and 70 or less, preferably 11 or more, 12 or more, or 13 or more, and preferably 60 or less, 50 or less, 40 or less, 30 or less, 24 or less, 22 or less, 20 or less, 18 or less, or 16 or less. From the viewpoint of editing activity, the number of residues of the 3′ oligonucleotide chain linked to the 3′ side of the target-corresponding nucleotide residue is 2 or more and 7 or less, and preferably 3 or more and 5 or less, 3 or more and 4 or less, or 3.

In the first oligonucleotide, the 3′-side oligonucleotide linked to the 3′ side of the target-corresponding nucleotide residue may have a complementary base sequence that does not contain a mismatched base pair to the target RNA. In that case, the target RNA preferably does not have a guanosine residue linked to the 5′ side of the adenosine residue that is the editing target. That is, when the nucleotide base linked to the 5′ side of the adenosine residue that is the editing target of the target RNA is an adenosine residue, a cytidine residue, or a uridine residue, it is preferable that in the first oligonucleotide, the oligonucleotide linked to the 3′ side of the target-corresponding nucleotide residue preferably has a base sequence complementary to the target RNA without a mismatched base pair.

In the first oligonucleotide, the 3′-side oligonucleotide linked to the 3′ side of the target-corresponding nucleotide residue may optionally contain a non-complementary base to the base sequence of the target RNA. For example, a target RNA in which a guanosine residue is linked to the 5′ side of an adenosine residue serving as an editing target may have a reduced activity of inducing editing. Even in that case, the editing-inducing activity can be improved by having the first oligonucleotide have a nucleotide sequence that includes a non-complementary base to the guanosine residue. A non-complementary base to the guanosine residue may be, for example, a guanosine residue. That is, a target editing guide RNA for a target RNA in which a guanosine residue is linked to the 5′ side of an adenosine residue serving as an editing target may have a guanosine residue linked to the 3′ side of the target-corresponding nucleotide residue.

The second oligonucleotide may consist of, for example, 2 or more and 30 or less nucleotide residues, and it may have a complementary or non-complementary base sequence to the corresponding sequence of the target RNA. The third oligonucleotide has a nucleotide sequence that can form a double strand with the second oligonucleotide. The double strand may form a complete complementary strand or an incomplete complementary strand containing at least one mismatched base. The mismatch base may form a mismatch base pair and may be a nucleotide residue (bulge) inserted into at least one of the two strands. In other words, the double strand comprising the second oligonucleotide and third oligonucleotide may contain at least one nucleotide residue that does not form a complementary base pair. The nucleotide residue that does not form a complementary base pair may be a nucleotide residue (bulge) that is present in one of the second or third oligonucleotides without a base pair due to the deletion of a nucleotide residue corresponding to the other nucleotide residue. Nucleotide residues that do not form complementary base pairs may be nucleotide residues present in both oligonucleotides of the second and third oligonucleotides (mismatched base pairs), where base pairs consisting of corresponding nucleotide residues are mismatched base pairs (non complementary base pairs) having nucleobases that are non-complementary to each other.

The number of residues of the second oligonucleotide is preferably 4 or more and 28 or less, 10 or more and 26 or less, or 18 or more and 26 or less. The number of residues of the third oligonucleotide may be preferably 4 or more and 28 or less, 10 or more and 26 or less, or 18 or more and 26 or less.

The second oligonucleotide may have a sequence that is non-complementary to the corresponding sequence of the target RNA. In such a case, the sequence of the second oligonucleotide may be appropriately selected according to the corresponding base sequence of the target RNA. For example, if the corresponding base of the target RNA is a pyrimidine base, a purine base or a pyrimidine base that does not form a base pair should be selected as the corresponding base of the second oligonucleotide, preferably a pyrimidine base. When the corresponding base of the target RNA is a purine base, a pyrimidine or purine base that does not form base pairs should be selected as the corresponding base of the second oligonucleotide, and a purine base is preferable. Specifically, when the corresponding base of the target RNA is cytosine (C), cytosine (C), uracil (U) or adenine (A) can be selected as the corresponding base of the second oligonucleotide, and C or U is preferable. When the corresponding base of the target RNA is uracil (U), uracil (U), cytosine (C) or guanine (G) can be selected as the corresponding base of the second oligonucleotide, and U or C is preferable. When the corresponding base of the target RNA is adenine (A), adenine (A), guanine (G) or cytosine (C) can be selected as the corresponding base of the second oligonucleotide, and A or G is preferable. When the corresponding base of the target RNA is guanine (G), adenine (A), guanine (G) or uracil (U) can be selected as the corresponding base of the second oligonucleotide, and A or G is preferable. Specific examples of the second oligonucleotides having sequences that are non-complementary to the corresponding sequences of the target RNA include sequences containing GGG, GG, GC, GA, GU, UC, UG, UA, UU, CG, CA, CU, CC, AG, AA, AC, AU, or such.

The number of nucleotide residue pairs in the double strand formed from the second oligonucleotide and the third oligonucleotide is, for example, 2 pairs or more, preferably 4 pairs or more, 6 pairs or more, 12 pairs or more, 16 pairs or more, or 18 pairs or more, and for example 30 pairs or less, preferably 24 pairs or less, 20 pairs or less, 16 pairs or less, 14 pairs or less, 9 pairs or less, 8 pairs or less, or 7 pairs or less. The double strand formed from the second oligonucleotide and the third oligonucleotide preferably contains guanine (G) and cytosine (C) from the viewpoint of the stability of the double-stranded structure. The ratio of GC pairs in the base pairs of the complementary strand portion is, for example, 30% or more, preferably 60% or more, 65% or more, or 68% or more. Further, the GC pair may be a GU pair containing uracil (U) capable of forming a base pair with guanine (G) by tautomerization instead of cytosine (C).

From the viewpoint of target editing activity, the base sequence of the second oligonucleotide comprises on the 3′ side of the first oligonucleotide, at least one selected from the group consisting of a sequence consisting of 2 or 3 consecutive guanines (GG or GGG), a sequence consisting of consecutive uracil and guanine (UG), and a sequence consisting of consecutive guanine, uracil and guanine (GUG).

The second oligonucleotide may have at its 3′ end, a nucleotide chain of any sequence that does not form a complementary strand with the third oligonucleotide, in which the number of residues is 1 or more and 50 or less, preferably 2 or more and 10 or less, or 2 or more and 7 or less. In addition, the third oligonucleotide may have at its 5′ end, a nucleotide chain of any sequence that does not form a complementary strand with the second oligonucleotide, in which the number of residues is 1 or more and 50 or less, preferably 2 or more and 10 or less, or 2 or more and 7 or less.

Examples of the base sequence of the second oligonucleotide are illustrated below together with the base sequence of the third oligonucleotide paired therewith, but are not limited thereto. Also, the base sequences of the second oligonucleotide and the third oligonucleotide may be interchanged.

TABLE 1

| | | | |
|---|---|---|---|
| Ex1 | Second Oligonucleotide | GGGUGGAAUAGUAUAACAAUAU | SEQ ID NO: 1 |
| | Third Oligonucleotide | AUGUUGUUAUAGUAUCCCACCU | SEQ ID NO: 2 |
| Ex2 | Second Oligonucleotide | GGGUGGAAUAGUAUAACAAUAUGCU | SEQ ID NO: 3 |
| | Third Oligonucleotide | GGAAAUGUUGUUAUAGUAUCCCACCU | SEQ ID NO: 4 |
| Ex3 | Second Oligonucleotide | CUGCCAUCAGUCGGCGUGGACUGUAG | SEQ ID NO: 5 |
| | Third Oligonucleotide | GGAACCAUGCCGACUGAUGGCAG | SEQ ID NO: 6 |
| Ex4 | Second Oligonucleotide | GGGUGGAAUA | SEQ ID NO: 7 |
| | Third Oligonucleotide | UAUCCCACCU | SEQ ID NO: 8 |
| Ex5 | Second Oligonucleotide | GGGUGG | — |
| | Third Oligonucleotide | CCACCU | — |
| Ex6 | Second Oligonucleotide | GGGUG | — |
| | Third Oligonucleotide | CACCU | — |
| Ex7 | Second Oligonucleotide | GGGU | — |
| | Third Oligonucleotide | ACCU | — |

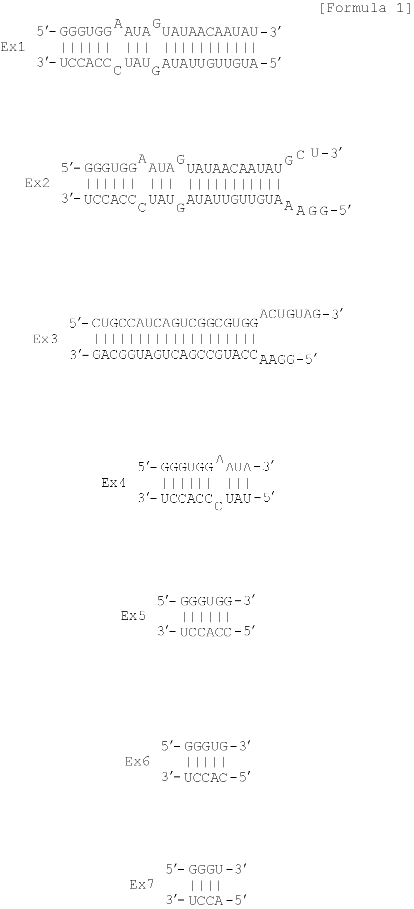

The second oligonucleotide and the third oligonucleotide may be linked by a second linkage group. As a result, the target editing guide oligonucleotide becomes a circular oligonucleotide as a whole and has improved resistance to hydrolysis by, for example, RNase or such. The second linkage group links the 3′ end of the second oligonucleotide and the 5′ end of the third oligonucleotide. As a result, a stem-loop structure is formed that includes a stem portion composed of the second oligonucleotide and the third oligonucleotide, and a loop portion composed of the second linkage group. The second linkage group may be, for example, a nucleotide chain with any arbitrary sequence of 4 or more residues and 12 or less residues. Specific examples of the sequence of the second linkage group include, for example, GCUAA; UNCG fold types such as UUCG, UACG, UGCG, and UCCG; GNRA fold types such as GAAA, GUAA, GCAA, GGAA, GAGA, GUGA, GCGA, and GGGA; RNYA fold type such as GUCA, GCCA, GGCA, GACA, AUCA, ACCA, AGCA, AACA, GUUA, GCUA, GGUA, GAUA, AUUA, ACUA, AGUA, AAUA; GGUG fold type; CUUG fold type; and AGNN fold types such as AGUU, AGUC, AGUG, AGUA, AGCU, AGCC, AGCG, and AGCA. For details of these loop structures, one can refer to, for example, Biophys. J., 113, 257-267, 2017.

The second oligonucleotide and the third oligonucleotide may contain a nucleotide sequence that enables intracellular expression of a circular type RNA. As a result, a circular-type target editing guide oligonucleotide can be expressed in cells using a plasmid or the like. For the method of expressing arbitrary circular RNA in a cell, one can refer to Nat. Biotechnol. 10, 1038 (2019) or such. A precursor circular RNA expressed by a plasmid capable of expressing a circular-type target editing guide oligonucleotide in cells, for example, may be constituted to comprise a 5′-ribozyme region (SEQ ID NO: 9) and a 5′-ligation stem region (SEQ ID NO: 10), a third oligonucleotide, a first linkage group, a first oligonucleotide, a second oligonucleotide, a 3′-ligation stem region (SEQ ID NO: 11), and a 3′-ribozyme region (SEQ ID NO: 12) in this order. In addition, the precursor circular RNA may further contain a linking region of any sequence of 1 or more residues and 23 or less residues between each functional region. For example, it is possible to link the 5′-ribozyme region together with the 5′-ligation stem region, and the 3′-ligation stem region together with the 3′-ribozyme region, by an arbitrary sequence of 2 or more residues and 8 or less residues capable of forming a second linkage group. Specifically, for example, two adenosine residues may be included between the 5′-ribozyme region and the 5′-ligation stem region. Also, for example, between the 3′-ligation stem region and the 3′-ribozyme region, 6 residues forming a complementary strand with the corresponding portion of the 3′-ribozyme region and G (for example, ACUGUAG) may be included. In the precursor circular RNA, the 5′-side and 3′-side ribozyme regions are autocatalytically cleaved, and the ligation reaction proceeds at the end of the stem structure consisting of the 5′-ligation stem region and the 3′-ligation stem region to form a circular RNA. At this time, if an arbitrary sequence is added to each end of the stem structure, the ligation reaction proceeds at the end of the arbitrary sequence to form a second linkage group.

In addition, the precursor circular RNA may comprise an arbitrary sequence capable of forming a double strand between the 5′-ligation stem region and the third oligonucleotide and between the second oligonucleotide and the 3′-ligation stem region, respectively. Arbitrary sequences may, for example, form regions that can interact with fluorochromes. Specifically, for example, it may be Broccoli RNA described in Angew. Chem. Int. Ed. Engl. 58, 1266-1279 (2019). Further, in the precursor circular RNA, the third oligonucleotide may be the 5′-ligation stem region, and the second oligonucleotide may be the 3′-ligation stem region.

TABLE 2

| | | |
|---|---|---|
| 5′-ribozyme region | GCCAUCAGUCGCCGGUCCCAAGCCCGGAUAAAAUGGGAGGG | SEQ ID NO: 9 |
| | GGCGGGAAACCGCCU | |
| 5′-ligation stem region | CCAUGCCGACUGAUGGCAG | SEQ ID NO: 10 |
| 3′-ligation stem region | CUGCCAUCAGUCGGCGUGG | SEQ ID NO: 11 |
| 3′-ribozyme region | AACACUGCCAAUGCCGGUCCCAAGCCCGGAUAAAAGUGGAG | SEQ ID NO: 12 |
| | GGUACAGUCCACGC | |

Also, the second linkage group may contain a molecular structure other than the nucleotide residues. Examples of a molecular structure other than the nucleotide residues include alkyleneoxy structural units.

Examples of base sequences formed by linking the second oligonucleotide, the second linkage group and the third oligonucleotide are shown below, but they are not limited thereto. In addition, Ex12 and Ex13 shown below contain a base sequence that enables the intracellular expression of circular type RNA.

TABLE 3

| | | |
|---|---|---|
| Ex11 | GGGUGGAAUAGUAUAACAAUAUGCUAAAUGUUG | SEQ ID NO: 13 |
| | UUAUAGUAUCCCACCU | |
| Ex12 | CUGCCAUCAGUCGGCGUGGACUGUAGAACCAUG | SEQ ID NO: 14 |
| | CCGACUGAUGGCAG | |
| Ex13 | CUGCCAUCAGUCGGCGUGGACUGUAGGGAACCA | SEQ ID NO: 15 |
| | UGCCGACUGAUGGCAG | |
| Ex14 | GGGUGGAAUAUUCGUAUCCCACCU | SEQ ID NO: 16 |
| Ex15 | GGGUGGUUCGCCACCU | SEQ ID NO: 17 |
| Ex16 | GGGUGUUCGCACCU | SEQ ID NO: 18 |
| Ex17 | GGGUUUCGACCU | SEQ ID NO: 19 |

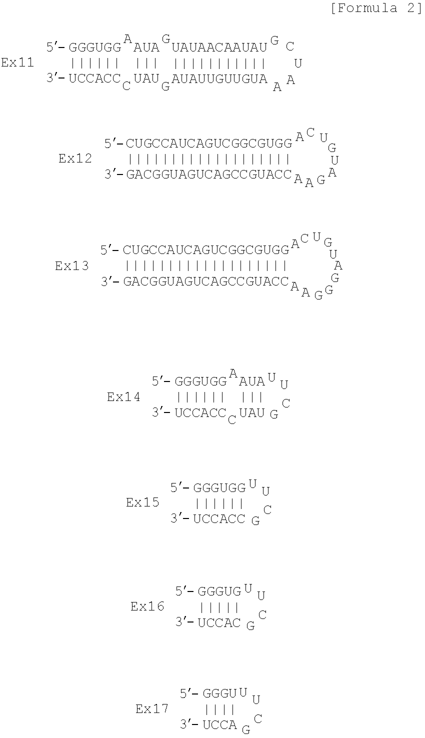

The first linkage group only needs to have the function of linking the first oligonucleotide and the third oligonucleotide, and may be a nucleotide chain of any arbitrary sequence. When the first linkage group consists of a nucleotide chain, the number of residues of the first linkage group may be, for example, 10 or more and 50 or less, preferably 20 or more and 50 or less. Further, the number of residues of the first linkage group may be, for example, 8 or more and 50 or less, preferably 8 or more and 30 or less, or 8 or more and 12 or less. The base sequence of the first linkage group is not particularly limited as long as it is a sequence that does not interfere with the target RNA. For example, it may be an oligonucleotide chain consisting of a single type of base, or an oligonucleotide chain consisting of a random base sequence. Also, the first linkage group may be consisted of a molecular structure other than nucleotides, such as an alkyleneoxy structural unit.

A target editing guide oligonucleotide comprising a first oligonucleotide that identifies a target RNA, a second oligonucleotide linked to the 3′ side of the first oligonucleotide, a third oligonucleotide having a base sequence capable of forming a complementary strand with the second oligonucleotide, and a first linkage group linking the 5′ end of the first oligonucleotide to the 3′ end of the third oligonucleotide can be chemically synthesized by a conventional method. Alternatively, for example, a template DNA is synthesized using an appropriate oligo-DNA pair in which the T7 promoter region can form a double strand, and a target editing guide oligonucleotide having a desired base sequence can be obtained by an in vitro transcription reaction.

The target editing guide oligonucleotide further comprising a second linkage group can be constructed by removing the 5′ end triphosphate from the 5′ end of the transcription product of an in vitro transcription reaction by a dephosphorylation reaction using alkaline phosphatase, then converting the 5′ end to a monophosphate by a phosphorylation reaction using polynucleotide kinase, followed by a ligation reaction using T4 RNA ligase or similar enzymes. In addition, during the in vitro transcription reaction, GMP (guanosine monophosphate) is added as a substrate in an in vitro transcription reaction to obtain a transcription product with the 5′ end in the form of a monophosphate, which can then be subjected to a ligation reaction to construct a second linkage group. Target-editing guide oligonucleotides further comprising the second linkage group may also be constructed by referring, for example, to the method of intracellular expression of any circular RNA described above.

The nucleotide residues constituting the target editing guide RNA may comprise a natural type ribonucleotide residue. That is, the target editing guide oligonucleotide may be a target editing guide RNA. The nucleotide residues constituting the target editing guide RNA may comprise a non-natural modified nucleotide residue. Modified nucleotide residues include those modified with a phosphodiester bond between nucleosides, those modified with a 2′ hydroxyl group of ribose, those containing an intramolecularly cross-linked ribose, and those modified with at least one of a purine base and a pyrimidine base. Examples of modification of the phosphodiester bond moiety include phosphorothioation, methylphosphonation, methylthiophosphonation, phosphorodithioation, phosphoramidation, peptide bond substitution and the like. Examples of modification of the 2′ hydroxyl group of ribose are 2′-O-methylation, 2′-O-methoxyethylation, 2′-O-aminopropyl (AP) conversion, 2′-fluoromation, 2′-O-methylcarbamoylethylation, and 3,3-dimethylallylation, and the like. Examples of an intramolecularly cross-linked ribose include nucleotides crosslinked at the 2′ position and 4′ position (2′, 4′-BNA). 2′, 4′-BNA includes, for example, locked nucleic acid (α-L-methyleneoxy (4′-CH₂—O-2′) BNA or ß-D-methyleneoxy (4′-CH₂—O-2′) BNA, which is also called LNA, ethyleneoxy (4′-(CH₂)₂—O-2′) BNA which is also known as ENA, ß-D-thio (4′-CH₂—S-2′) BNA, aminooxy (4′-CH₂—O—N(R)-2′) BNA (R is H or CH₃), oxyamino (4′-CH₂—N(R)—O-2′) BNA (R is H or CH₃) which is also called 2′, 4′-BNANC, 2′, 4′-BNACOC, 3′-amino-2′, 4′-BNA, 5′-methyl BNA, (4′-CH(CH₃)—O-2′) BNA which is also called cEt-BNA, (4′-CH(CH₂OCH₃)—O-2′) BNA which is also called cMOE-BNA, amide type BNA (4′-C(O)—N(R)-2′) BNA (R is H or CH₃) which is called AmNA, and the like. Examples of modification of the base moiety include halogenation; alkylation such as methylation, ethylation, n-propylation, isopropylization, cyclopropylation, n-butylation, isobutylation, s-butylation, t-butylation, cyclobutylation; hydroxylation; amination; deaminolation; demethylation and the like.

### Site-Specific Target RNA Editing Method

A method for site-specific editing of a target RNA includes contacting the target RNA with a target editing guide oligonucleotide, which is an oligonucleotide that induces site-specific editing of the target RNA, in the presence of adenosine deaminase. The target-editing guide oligonucleotide forms a partial double strand with the target RNA and recruits adenosine deaminase in a site-specific manner to convert an adenosine residue contained in the target RNA into an inosine residue.

The site-specific target RNA editing method can be carried out, for example, by introducing or expressing the above-described target editing guide oligonucleotide into a eukaryotic cell having the target RNA. The method for introducing the target editing guide RNA into a eukaryotic cell can be appropriately selected and applied from various methods used in nucleic acid medicine. In addition, by introducing a plasmid or the like capable of expressing the target editing guide oligonucleotide into the eukaryotic cell, the target editing guide oligonucleotide can be expressed in the eukaryotic cell. The method of site-specific editing of a target RNA can be performed in vitro or in vivo.

By applying the method of site-specific target RNA editing with the use of a target editing guide oligonucleotide to mutations of amino acids that are involved in functional expression of intracellular proteins such as sugar chain modification sites, phosphorylation sites, and metal coordination sites, it will be possible to provide a new methodology for temporally controlling intracellular protein functions. In addition, by generalizing the method of controlling the function of proteins in vivo by site-specific editing of target RNA using target editing guide oligonucleotides, this molecular technology can contribute to the development of research in the field of life sciences.

Conventionally, nucleic acid medicines have been developed that use suppression of target protein expression by siRNA or functional control of target proteins by functional RNA called aptamers. However, there is no example of a drug that converts mRNA information and modifies the function of the target protein encoded by the mRNA. Therefore, target editing guide oligonucleotides have the potential to create novel nucleic acid drugs that exhibit unprecedented efficacy.

For example, a nonsense mutant hereditary disease is a disease caused by the failure to synthesize the original protein due to a stop codon formed by a point mutation on the gene. Nonsense mutant hereditary diseases include, for example, muscular dystrophy, multiple sclerosis, Alzheimer's disease, neurological tissue degeneration, neurological diseases such as Parkinson's disease, and cancer. For example, by editing stop codons such as UAA, UAG, and UGA with a target editing guide RNA, it can be used as a nucleic acid drug having an unprecedented mechanism for the above-mentioned diseases. Specifically, for example, it is conceivable to control protein synthesis by editing UAG, which is a stop codon, into UIG, which is a tryptophan codon.

Also, for example, many proteins that have important intracellular functions are precisely regulated ON/OFF by phosphorylation and dephosphorylation, and it has been suggested that abnormalities in phosphorylation and dephosphorylation are deeply involved in various diseases including cancer. Protein phosphorylation sites include Tyr, Thr, Ser, and such. By editing codons encoding these amino acids to codons encoding other amino acids by a target editing guide RNA, it is possible to inhibit the phosphorylation of proteins. Specifically, for example, by editing the Tyr codon, UAC, to UIC, which is a Cys codon, the phosphorylation of intracellular proteins can be regulated.

In the target editing guide oligonucleotide, the 5′ end of the first oligonucleotide and the 3′ end of the third oligonucleotide are linked by the first linkage group. Further, the second oligonucleotide and the third oligonucleotide form a double-stranded structure, resulting in a ring-substituted structure (pseudo-cyclic structure) as a whole. In some cases, the 3′ end of the second oligonucleotide and the 5′ end of the third oligonucleotide are linked by a second linkage group to form a cyclic structure as a whole. Although nucleic acids are generally linear, it is known that their intracellular stability can be improved by protecting the ends that are susceptible to degradation in the cell. In other words, the formation of a structure such as a double stranded structure at the end improves resistance to degradation. Also, similarly, the intracellular stability of nucleic acids without ends, i.e., cyclic nucleic acids, is dramatically improved compared to that of general linear nucleic acids. Since the target editing guide oligonucleotide has a circular or cyclic structure, its resistance to degradation in vivo is improved and it can exhibit a higher intracellular editing induction activity, compared to conventional target editing guide RNAs constructed with linear nucleic acids.

### EXAMPLES

The present invention will be specifically described below by way of examples, but the present invention is not limited to these examples.

### Reference Example 1

### Preparation of Model Target RNA

The GFP-Gq-TK Plasmid was used as a template for amplification by PCR (1 cycle (98° C., 10 seconds; 55° C., 30 seconds; 68° C., 30 seconds), 30 cycles), in a reaction solution comprising 100 μM AcGFP_sRNA02_T7F01 primer (SEQ ID NO: 20), 100 μM AcGFP_sRNA02_R01 primer (SEQ ID NO: 21), 2.5 mM dNTP, 1.25 U/mL Prime Star GXL (manufactured by Takara Bio) (final concentrations: GFP-Gq-TK Plasmid 4.0 pg/mL, AcGFP_sRNA02_T7 F/R 0.3 M, dNTP 0.2 mM, PrimeStar GXL 1.25 U). DNA was purified from the amplified PCR products by phenol/chloroform extraction and ethanol precipitation. Using the obtained DNA as a template, RNA was synthesized by in vitro transcription (37° C., 3 hours) using the T7-Scribe Standard RNA IVT KIT (manufactured by CELLSCRIPT). Then, DNase (final concentration: 2 U) was added for treatment (37° C., 30 minutes), and RNA was purified by phenol/chloroform extraction and ethanol precipitation. The resulting RNA was purified by 8M Urea PAGE (8%), extracted by grinding and immersion, and purified by 0.22 mm filter (manufactured by DURAPORE) and gel filtration (manufactured by BIO-RAD) to prepare a model target RNA of 160 nt residues (GFP A200) (SEQ ID NO: 22). The primers used and the resulting model target RNA sequences are shown in Table 4. The underlined bold part is the editing target adenosine residue.

TABLE 4

| | | |
|---|---|---|
| AcGFP_sRNA02_T7F01 | CTAATACGACTCACTATAGGGTGAATGGCCACAAGTTCAG | SEQ ID NO: 20 |
| AcGFP_sRNA02_R01 | TAGCGTGAGAAGCACTGCAC | SEQ ID NO: 21 |
| GFP A200 | GGGUGAAUGGCCACAAGUUCAGCGUGAGCGGCGAGGGCGAG | SEQ ID NO: 22 |
| | GGCGAUGCCACCUACGGCAAGCUGACCCUGAAGUUCAUCUG | |
| | CACCACCGGCAAGCUGCCUGUGCCCUGGCCCAGGCUGGUGA | |
| | CCACCCUGAGCUACGGCGUGCAGUGCUUCUCACGCUA | |

### Reference Example 2

With reference to the description of Example 1 of WO 2017/010556, an ADAR binding region consisting of 49 residues forming a stem loop structure on the 3′ side of the antisense region is linked, a conventional target editing guide RNA (hereinafter also referred to as ADg_GFP200) (SEQ ID NO: 23) was prepared. Table 4 shows the sequence of the target editing guide RNA obtained. In Table 5, the bold underlined part is the target corresponding nucleotide residue.

TABLE 5

| | | |
|---|---|---|
| ADg_GFP200 | GGAAGCACUGGACGCCGCAGGGGGUGGAAUAGUAUAACAAU | SEQ ID NO: 23 |
| | AUGCUAAAUGUUGUUAUAGUAUCCCACCU | |

ADg_GFP200 is composed of a first oligonucleotide that identifies a target RNA and an oligonucleotide capable of forming a stem-loop structure linked to its 3′ side, as shown in the schematic structure below.

### Example 1

Preparation of cpADg_L10Glu_GFP200

For a solution comprising 100 μM 5ASg_split_GFP200F oligo DNA (SEQ ID NO: 24) and 100 μM 5ASg_split_GFP200R oligo DNA (SEQ ID NO: 25), an annealing reaction was carried out by 3 minutes of heat denaturation at 95° C. and 15 minutes of cooling to 25° C. After that, 2.5 mM dNTP and 5000 U/mL of the Klenow Fragment (manufactured by New England Biolabs) were added, and an extension reaction was carried out at 25° C., for 30 minutes (final concentrations: oligo DNA 2 μM, dNTP 0.2 mM, Klenow Fragment 2.5 U). Following the reaction, DNA was purified by phenol/chloroform extraction and ethanol precipitation. The obtained DNA was used as a template and RNA was synthesized by in vitro transcription (37° C., 3 hours) using the T7-Scribe Standard RNA IVT KIT (manufactured by CELLSCRIPT). Then, DNase (final concentration: 2 U) was added for treatment (37° C., 30 minutes), and RNA was purified by phenol/chloroform extraction and ethanol precipitation. The obtained RNA was purified by 8M Urea PAGE (8%), extracted by the crush-and-soak method, and purified by 0.22 mm filter (manufactured by DURAPORE) and gel filtration (manufactured by BIO-RAD) to prepare a target editing guide RNA (cpADg_L10Glu_GFP200) (SEQ ID NO: 26) of 80 nt residues. Table 6 shows the primers used and the resulting target editing guide RNA sequence.

TABLE 6

| | | |
|---|---|---|
| 5ASg_split_GFP200F | CTAATACGACTCACTATAGGAAATGTTGTTATAGTATCCGA | SEQ ID NO: 24 |
| | CCTTTTTTTTTTTAAGCACTGC | |
| 5ASg_split_GFP200R | AGCATATTGTTATACTATTCCACCCGCTGCGGCGTGCAGTG | SEQ ID NO: 25 |
| | CTTAAAAAAAAAAAGG | |
| cpADg_L10Glu_GFP200 | GGAAAUGUUGUUAUAGUAUCCCACCUUUUUUUUUUUAAGCA | SEQ ID NO: 26 |
| | CUGCACGCCGCAGCGGGUGGAAUAGUAUAACAAUAUGCU | |

cpADg_L10Glu_GFP200 is composed of a third oligonucleotide, a first linkage group (L10) consisting of 10 U residues, a first oligonucleotide, and a second oligonucleotide, as shown in the schematic structure below. The second oligonucleotide and the third oligonucleotide form a double strand.

### Example 2

Preparation of cpADg_L30Glu_GFP200

An annealing reaction was carried out by heat denaturation at 95° C., for 3 minutes in a solution comprising 100 μM v5AS_Glu_GFP_5F01 oligo DNA (SEQ ID NO: 27) and 100 μM v5AS_Glu_GFP_5 R01 oligo DNA (SEQ ID NO: 28), followed by 15 minutes of cooling to 25° C. After that, 2.5 mM of dNTP. 5000 U/mL of the Klenow Fragment (manufactured by New England Biolabs) was added, and an extension reaction was carried out at 25ºC for 30 minutes to prepare 5′ side fragment DNA (final concentrations: oligo DNA 2 μM, dNTP 0.2 m.M. Klenow Fragment 2.5 U). For a solution comprising 100 μM v5AS_Glu_GFP_3F01 oligo DNA (SEQ ID NO: 29) and 100 μM v5AS_Glu_GFP_3 R01 oligo DNA (SEQ ID NO: 30), an annealing reaction was carried out by 3 minutes of heat denaturation at 95° C., and 15 minutes of cooling to 25° C. After that, 2.5 mM of dNTP, 5000 U/mL of the Klenow Fragment (manufactured by New England Biolabs) was added, and an extension reaction was carried out at 25ºC for 30 minutes to prepare 3′ side fragment DNA (final concentrations: oligo DNA 2 μM, dNTP 0.2 mM, Klenow Fragment 2.5 U). Amplification by PCR (1 cycle (98° C., 10 seconds; 55° C., 30 seconds; 68° C., 20 seconds), 30 cycles) using each DNA fragment as a template was performed in a reaction solution comprising 100 M of the T7proGG primer (SEQ ID NO: 31), 100 M of the 5AS_Glu_cp_R01 primer (SEQ ID NO: 32), 2.5 mM of dNTPs, and 1.25 U/mL Prime Star GXL (manufactured by Takara Bio Inc.) (final concentrations: T7proGG 0.3 μM, 5AS_Glu_cp_R01 0.3 μM, dNTP 0.2 mM, PrimeStar GXL 1.25 U). DNA was purified from the amplified PCR products by phenol/chloroform extraction and ethanol precipitation. The obtained DNA was used as a template to synthesize RNA by in vitro transcription (37° C. 3 hours) using the T7-Scribe Standard RNA IVT KIT (manufactured by CELLSCRIPT). Then, DNase (final concentration: 2 U) was added for treatment (37° C., 30 minutes), and RNA was purified by phenol/chloroform extraction and ethanol precipitation. The resulting RNA was purified by 8M Urea PAGE (8%), extracted by the crush-and-soak method, and purified by 0.22 mm filter (manufactured by DURAPORE) and gel filtration (manufactured by BIO-RAD) to prepare a target editing guide RNA of 100 nt residues (cpADg_L30Glu_GFP200) (SEQ ID NO: 33). Table 7 shows the primers used and the resulting target editing guide RNA sequence.

TABLE 7

| | | |
|---|---|---|
| v5AS_Glu_GFP_5F01 | CTAATACGACTCACTATAGGAAATGTTGTTATAGTATCCCA | SEQ ID NO: 27 |
| | CCTCGACGTTG | |
| v5AS_Glu_GFP_5R01 | GTGCTTGAATTCACTGGCCGTCGTTTTACAACGTCGAGGTG | SEQ ID NO: 28 |
| | GGATAC | |
| v5AS_Glu_GFP_3F01 | GACGGCCAGTGAATTCAAGCACTGCACGCCGCAGCGGGTGG | SEQ ID NO: 29 |
| v5AS_Glu_GFP_3R01 | AGCATATTGTTATACTATTCCACCCGCTGCGGCGTGCAG | SEQ ID NO: 30 |
| T7proGG | CTAATACGACTCACTATAGG | SEQ ID NO: 31 |
| 5AS_Glu_cp_R01 | AGCATATTGTTATACTATTCCACC | SEQ ID NO: 32 |
| cpADg_L30Glu_GFP200 | GGAAAUGUUGUUAUAGUAUCCCACCUCGACGUUGUAAAACG | SEQ ID NO: 33 |
| | ACGGCCAGUGAAUUCAAGCACUGCACGCCGCAGGGGGUGGA | |
| | AUAGUAUAACAAUAUGCU | |

cpADg_L30Glu_GFP200 is composed of a third oligonucleotide, a first linkage group (L30) consisting of a random sequence of 30 residues, a first oligonucleotide, and a second oligonucleotide, as shown in the schematic structure below, The second oligonucleotide and the third oligonucleotide form a double strand.

### Example 3

Preparation of cpADg_L30LgST_GFP200

An annealing reaction was performed by heat denaturation at 95° C., for 3 minutes in a solution comprising 20 μM of 5STEMg_GFP_V30_FW oligo DNA (SEQ ID NO: 34) and 20 μM of 5STEMg_GFP_V30_RV oligo DNA (SEQ ID NO: 35), followed by 15 minutes of cooling to 25° C. After that, 2.5 mM of dNTP, 5000 U/mL of the Klenow Fragment (manufactured by New England Biolabs) was added, and an extension reaction was carried out at 25ºC for 30 minutes (final concentrations: oligo DNA 0.4 μM, dNTP 0.2 mM, Klenow Fragment 2.5 U). DNA was purified from the amplified PCR products by phenol/chloroform extraction and ethanol precipitation. The obtained DNA was used as a template to synthesize RNA by in vitro transcription (37° C., 3 hours) using the T7-Scribe Standard RNA IVT KIT (manufactured by CELLSCRIPT). Then, DNase (final concentration: 2 U) was added for treatment (37° C., 30 minutes), and RNA was purified by phenol/chloroform extraction and ethanol precipitation. The resulting RNA was purified by 8M Urea PAGE (8%), extracted by the crush-and-soak method, and purified by 0.22 mm filter (manufactured by DURAPORE) and gel filtration (manufactured by BIO-RAD) to prepare a target editing guide RNA of 98 nt residues (cpADg_L30LgST_GFP200) (SEQ ID NO: 36). Table 8 shows the primers used and the resulting target editing guide RNA sequence.

TABLE 8

| | | |
|---|---|---|
| 5STEMg_GFP_V30_FW | CTAATACGACTCACTATAGGAACCATGCCGACTGATGGCAG | SEQ ID NO: 34 |
| | CGACGTTGTAAAACGACGGCCAGTGAATTCAAGCAC | |
| 5STEMg_GFP_V30_RV | CTACAGTCCACGCCGACTGATGGCAGGCTGCGGCGTGCAGT | SEQ ID NO: 35 |
| | GCTTGAATTCACTGGCCGTCG | |
| cpADg_L30LgST_GFP200 | GGAACCAUGCCGACUGAUGGCAGCGACGUUGUAAAACGACG | SEQ ID NO: 36 |
| | GCCAGUGAAUUCAAGCACUGCACGCCGCAGCCUGCCAUCAG | |
| | UCGGCGUGGACUGUAG | |

cpADg_L10LgST_GFP200 is composed of a third oligonucleotide, a first linkage group (L30) consisting of a random sequence of 30 residues, a first oligonucleotide, and a second oligonucleotide, as shown in the schematic structure below. The structural region corresponding to ARR consisting of the second oligonucleotide and the third oligonucleotide in cpADg_L30LgST_GFP200 is a nucleotide sequence that forms a complete double stranded structure without mismatches, and is a base sequence that enables circularization reaction in cells.

### Comparative Example 1

### Preparation of ASR-Linker_GFP200

An annealing reaction was performed by heat denaturation at 95ºC for 3 minutes in a solution comprising 20 μM of cp5AS_GFP_vect30_F oligo DNA (SEQ ID NO: 37) and 20 μM of cp5AS_GFP_vect30_R oligo DNA (SEQ ID NO: 38), followed by 15 minutes of cooling to 25° C. After that, 2.5 mM of dNTP, 5000 U/mL of the Klenow Fragment (manufactured by New England Biolabs) was added, and an extension reaction was carried out at 25ºC for 30 minutes (final concentrations: oligo DNA 0.4 M, dNTP 0.2 mM, Klenow Fragment 2.5 U). DNA was purified from the amplified PCR product by phenol/chloroform extraction and ethanol precipitation. The obtained DNA was used as a template to synthesize RNA by in vitro transcription (37° C., 3 hours) using the T7.Scribe Standard RNA IVT KIT (manufactured by CELLSCRIPT). Then, DNase (final concentration: 2 U) was added for treatment (37° C., 30 minutes), and RNA was purified by phenol/chloroform extraction and ethanol precipitation. The resulting RNA was purified by 8M Urea PAGE (8%), extracted by the crush and soak method, and purified by 0.22 mm filter (manufactured by DURAPORE) and gel filtration (manufactured by BIO-RAD) to prepare a target editing guide RNA of 51 nt residues (ASR-Linker_GFP200) (SEQ ID NO: 39). Table 9 shows the primers used and the resulting target editing guide RNA sequence.

TABLE 9

| | | |
|---|---|---|
| cp5AS_GFP_vect30_F | CTAATACGACTCACTATAGGCGACGTTGTAAAACGACGGCC | SEQ ID NO: 37 |
| | AGTGAATTCAAG | |
| cp5AS_GFP_vect30_R | GCTGCGGCGTGCAGTGCTTGAATTCACTGGCCGTCG | SEQ ID NO: 38 |
| ASR-Linker_GFP200 | GGCGACGUUGUAAAACGACGGCCAGUGAAUUCAAGCACUGC | SEQ ID NO: 39 |
| | ACGCCGCAGC | |

ASR.Linker_GFP200 is composed of an oligonucleotide (L30) consisting of a random sequence of 30 residues and a first oligonucleotide, as shown in the schematic structure below.

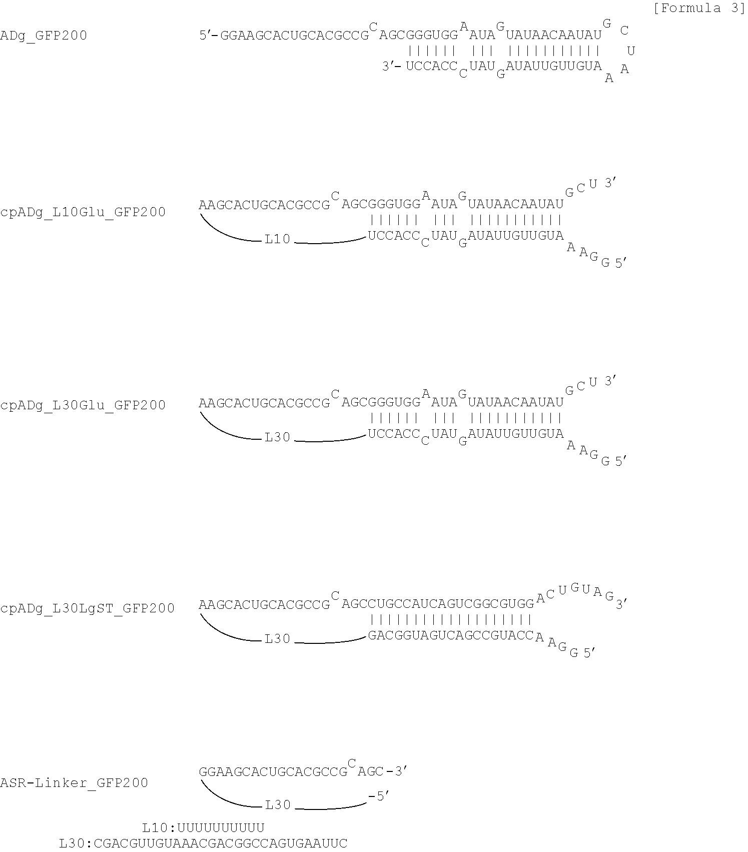

### Evaluation 1

The editing inducing activity of the target editing guide RNA (gRNA) prepared above was evaluated in vitro using a model target RNA (GFP A200). First, model target RNA and target editing guide RNA were subjected to an annealing reaction to form a complex, and purified recombinant hADAR2 was added to perform an editing reaction. To analyze the editing efficiency of the target site, the cDNA of the target RNA was amplified by RT-PCR, and the editing ratio was calculated from the chromatogram obtained by direct sequencing. A specific protocol is as follows.

Evaluation of the Editing Induction Ability of a Target Editing Guide RNA (gRNA) (in vitro)

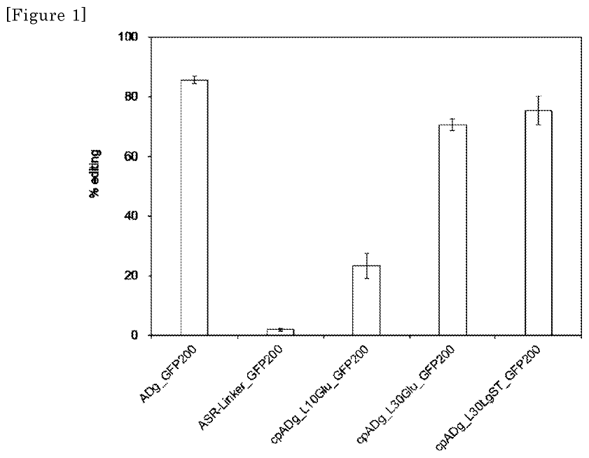

An annealing reaction was performed in an annealing buffer (150 mM NaCl, 10 mM Tris-HCl (pH 7.6)) comprising 0.3 μM of model target RNA and 0.9 μM of gRNA by heat denaturation at 80° C., for 3 minutes and cooling to 25° C., for 15 minutes. An editing reaction was carried out for 5 nM of the RNA complex and 10 nM of hADAR2 at 37ºC for 30 minutes in an editing reaction buffer (20 mM HEPES-KOH [pH 7.5], 100 mM NaCl, 2 mM MgCl2, 0.5 mM DTT, 0.01% Triton X-100, 5% glycerol, 1 U/μL Murine RNase Inhibitor (manufactured by New England BioLabs). After the reaction, RNA was purified by phenol/chloroform extraction and ethanol precipitation, and dissolved in 5 μL of TE buffer. The recovered RNA sample was subjected to reverse transcription using PrimeScript Reverse Transcriptase II (manufactured by Takara) to synthesize cDNA. Using the resulting cDNA as a template, dsDNA was amplified by PCR using 0.3 μM of the T7GFP_sRNA_F01 primer (SEQ ID NO: 40) and 0.3 μM of the GFP_sRNA_R01 primer (SEQ ID NO: 41). Direct sequencing of dsDNA amplified with 0.165 μM T7proGGG primer (SEQ ID NO: 42) was performed using the Big Dye Terminator v3.1 Cycle Sequencing Kit. Finally, the editing ratio (%) was calculated from the peak height ratio G/(G+A) of the obtained chromatographic chart. The results are shown in Table 11 and FIG. 1.

TABLE 10

| | | |
|---|---|---|
| T7GFP_sRNA_F01 | CTAATACGACTCACTATAGGGTGAATGGCCACAAGTTCAG | SEQ ID NO: 40 |
| GFP_sRNA_R01 | TAGCGTGAGAAGCACTGCAC | SEQ ID NO: 41 |
| T7proGGG | CTAATACGACTCACTATAGGG | SEQ ID NO: 42 |

TABLE 11

Editing ratio (%)

| gRNA | Mean (n = 3) | S.D. (n = 3) |
|---|---|---|
| ADg_GFP200 | 85.6 | 1.2 |
| cpADg_L10Glu_GFP200 | 23.2 | 4.2 |
| cpADg_L30Glu_GFP200 | 70.6 | 2.0 |
| cpADg_L30LgST_GFP200 | 75.3 | 4.9 |
| ASR-Linker_GFP200 | 1.8 | 0.4 |

All of cpADg_L10Glu_GFP200, cpADg_L30Glu_GFP200, and cpADg_L30LgST_GFP200 exhibited edit-inducing activity. In particular, cpADg_L30Glu_GFP200 and cpADg_L30LgST_GFP200 exhibited almost the same level of editing-inducing activity as the conventional type. On the other hand, ASR-Linker_GFP200 showed almost no editing-inducing activity.

### Example 4-1

All of cpADg_L10Glu_GFP200, cpADg_L30Glu_GFP200, and cpADg_L30LgST_GFP200 exhibited edit-inducing activity. In particular, cpADg_L30Glu_GFP200 and cpADg_L30LgST_GFP200 exhibited almost the same level of editing-inducing activity as the conventional type. On the other hand, ASR-Linker_GFP200 showed almost no editing-inducing activity.

For 100 pmol of cpADg_L30LgST_GFP200, a dephosphorylation reaction was carried out at 37º C for 30 minutes using 1 U of Antarctic Phosphatase (manufactured by New England Biolabs). RNA was then purified by phenol/chloroform extraction and ethanol precipitation. After drying, it was dissolved in 14 μL of dH₂O and incubated at 65º C for 10 minutes.

10 mM ATP and 20 U of T4 Polynucleotide Kinase (manufactured by Takara Bio Inc.) were added to the solution after dephosphorylation, and a phosphorylation reaction was performed at 37ºC for 30 minutes. RNA was then purified by phenol/chloroform extraction and ethanol precipitation. It was dissolved in 40 μL of TE buffer, and RNA was purified using Micro Bio-spin Columns P-30 (manufactured by BIO-RAD). 100 pmol of phosphorylated RNA was subjected to an RNA annealing reaction (95° C., 3 minutes) in an environment of 150 mM NaCl, 10 mM TrisHCI (pH 7.6), and then cooled to 25° C., for 15 minutes. The solution after annealing was reacted with 25% PEG6000, 0.006% BSA, and 60 U of T4 RNA ligase (manufactured by Takara Bio Inc.) at 10° C., for 16 hours. After that, RNA was then purified by phenol/chloroform extraction and ethanol precipitation. The gel excision purification using denaturing PAGE were then performed.

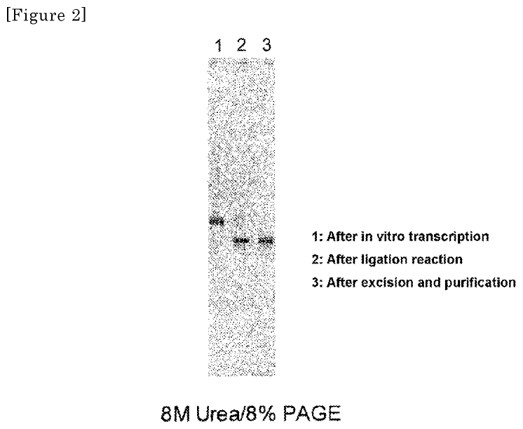

After the in vitro transcription, after the ligation reaction, and after the gel excision purification, each sample was subjected to 8M urea denatured polyacrylamide gel electrophoresis (denatured PAGE) to confirm the progress of the circularization reaction. The results are shown in FIG. 2.

In general, in the case of nucleic acids having the same base length, the circular form is observed as a band in the lower molecular weight side (higher mobility) when compared to the linear form. In circADg_L30LgST_GFP200 obtained by the above reaction, a band was observed on the lower molecular weight side compared to cpADg_L30LgST_GFP200 before the reaction, and thus a circular target-editing guide RNA, whose schematic structure is shown below, could be synthesized.

### Example 4-2

### RNA Circularization by GMP-Tagged IVT

An in vitro transcription reaction was performed using the T7-Scribe Standard RNA IVT Kit (CELL SCRIPT). ATP. CTP, UTP and GMP (manufactured by Nacalai Tesque Inc.) at a final concentration of 10 mM and 1 mM of GTP were added to 1 μg of template DNA, and 20 μL of the mixture was allowed to react at 37º C for 3 hours. RNA was then purified by phenol/chloroform extraction and ethanol precipitation. The resulting RNA was purified by 8M Urea PAGE (8%), extracted by the crush-and-soak method, purified by 0.22 mm filter (manufactured by DURAPORE) and gel filtration (manufactured by BIO-RAD), and circularized. A target editing guide RNA (circADg_L30LgST_GFP200) was prepared.

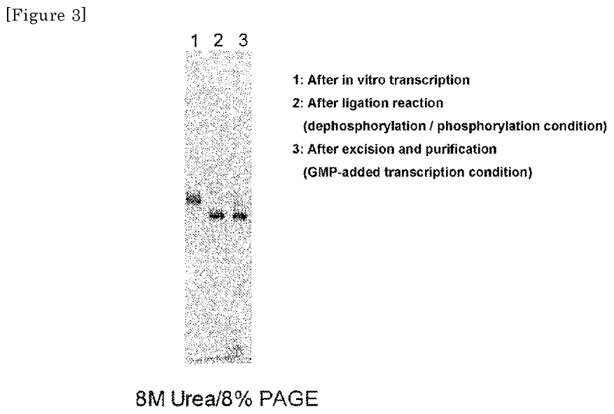

After the in vitro transcription, after the ligation reaction (dephosphorylation/phosphorylation conditions), and after the ligation reaction (GMP-added transcription condition), each sample was subjected to 8M urea denatured polyacrylamide gel electrophoresis (denatured PAGE) to confirm progress of the circularization reaction. The results are shown in FIG. 3.

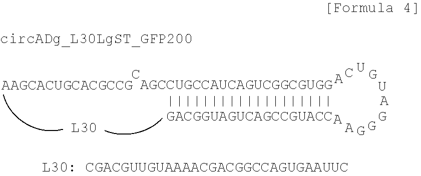

### Evaluation 2-1

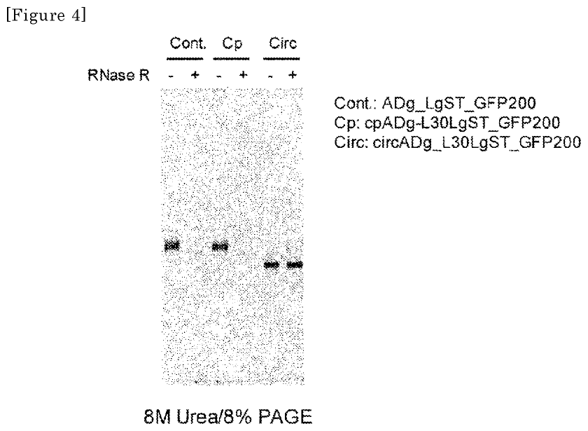

Degradation resistance was evaluated by reacting RNaseR (3′→5′ exoribonuclease) with the control (linear type: ADg_LgST_GFP200), cp type (cpADg-L30LgST_GFP200), and circ type (circADg_L30LgST_GFP200) RNA samples. Specifically, 1 U RNase R (manufactured by Lucigen) was added to 250 ng of the RNA sample, and the mixture was reacted at 37ºC for 1 hour. For the 50 ng of solution after the reaction, its degradation state was confirmed by 8M Urea PAGE (8%). The results are shown in FIG. 4.

The control and cp type were almost completely degraded. On the other hand, the circ type was not degraded at all. The above results indicated that circADg_L30LgST_GFP200 has an extremely high degradation resistance to exoribonuclease.

### Evaluation 2-2

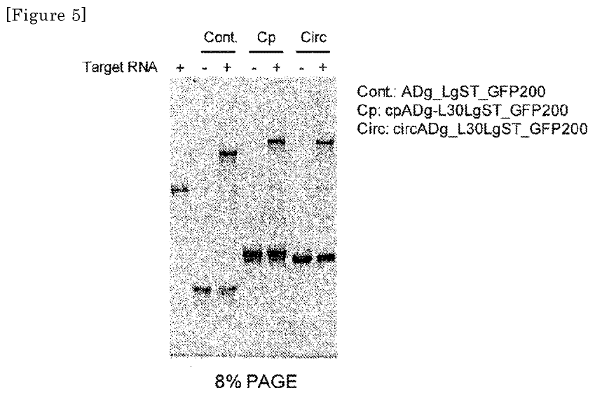

The control (ADg_LgST_GFP200), cp type (cpADg-L30LgST_GFP200), and circ type (circADg_L30LgST_GFP200) AD-gRNAs were subjected to an annealing reaction by adding target RNA (GFP A200), and after that, the mobility of the bands was then confirmed by native PAGE. The results are shown in FIG. 5.

A band indicating the complex was observed in all samples including the control. The above results indicate that circADg_L30LgST_GFP200 can form a complex with target RNA, similar to the control and cp type.

### Evaluation 2-3

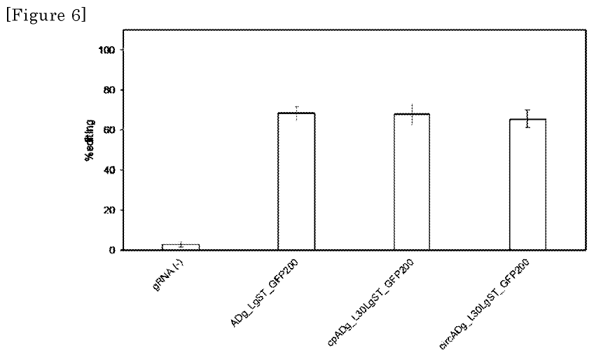

The editing induction activity was evaluated in the same manner as in Evaluation 1, except that ADg_LgST_GFP200, cpADg_L30LgST_GFP200, and circADg_L30LgST_GFP200 were used as target editing guide RNAs. The results are shown in Table 12 and FIG. 6.

TABLE 12

Editing Ratio (%)

| gRNA | Mean (n = 3) | S.D. (n = 3) |
|---|---|---|
| — | 2.9 | 1.2 |
| ADg_LgST_GFP200 | 68.2 | 3.5 |
| cpADg_L30LgST_GFP200 | 68.1 | 5.4 |
| circADg_L30LgST_GFP200 | 65.6 | 4.4 |

The circular-type (circ-type) target editing guide RNA exhibited an editing induction activity equivalent to that of the cp type, which is the form before circularization, and the linear type (ADg_LgST_GFP200). From the above results, it was shown that in in vitro editing induction, the circ-type target-editing guide RNA has an editing induction activity equivalent to that of a conventional type linear target-editing guide RNA.

### Reference Example 3

A target RNA (Rluc_W104X) (SEQ ID NO: 43) for the luciferase reporter assay was prepared with reference to the description in Example 23 of WO2019/111957. Rluc_W104X was obtained by converting 104W (tryptophan) in the region encoding Renilla luciferase (Rluc) to 104X (termination codon) and 41K (lysine) to 41R (arginine). Specifically, the editing target was set by mutating 311G, which corresponds to the 104th tryptophan, to 311A.

TABLE 13

| | | |
|---|---|---|
| Rluc_W104X | AUGGCUUCCAAGGUGUACGACCCCGAGCAACGCAAACGCAU | SEQ ID NO: 43 |
| | GAUCACUGGGCCUCAGUGGUGGGCUCGCUGCAAGCAAAUGA | |
| | ACGUGCUGGACUCCUUCAUCAACUACUAUGAUUCCGAGAGG | |
| | CACGCCGAGAACGCCGUGAUUUUUCUGCAUGGUAACGCUGC | |
| | CUCCAGCUACCUGUGGAGGCACGUCGUGCCUCACAUCGAGC | |
| | CCGUGGCUAGAUGCAUCAUCCCUGAUCUGAUCGGAAUGGGU | |
| | AAGUCCGGCAAGAGCGGGAAUGGCUCAUAUCGCCUCCUGGA | |
| | UCACUACAAGUACCUCACCGCUUAGUUCGAGCUGCUGAACC | |
| | UUCCAAAGAAAAUCAUCUUUGUGGGCCACGACUGGGGGGCU | |
| | UGUCUGGCCUUUCACUACUCCUACGAGCACCAAGACAAGAU | |
| | CAAGGCCAUCGUCCAUGCUGAGAGUGUCGUGGACGUGAUCG | |
| | AGUCCUGGGACGAGUGGCCUGACAUCGAGGAGGAUAUCGCC | |
| | CUGAUCAAGAGCGAAGAGGGCGAGAAAAUGGUGCUUGAGAA | |
| | UAACUUCUUCGUCGAGACCAUGCUCCCAAGCAAGAUCAUGC | |
| | GGAAACUGGAGCCUGAGGAGUUCGCUGCCUACCUGGAGCCA | |
| | UUCAAGGAGAAGGGCGAGGUUAGACGGCCUACCCUCUCCUG | |
| | GCCUCGCGAGAUCCCUCUCGUUAAGGGAGGCAAGCCCGACG | |
| | UCGUCCAGAUUGUCCGCAACUACAACGCCUACCUUCGGGCC | |
| | AGCGACGAUCUGCCUAAGAUGUUCAUCGAGUCCGACCCUGG | |
| | GUUCUUUUCCAACGCUAUUGUCGAGGGAGCUAAGAAGUUCC | |
| | CUAACACCGAGUUCGUGAAGGUGAAGGGCCUCCACUUCAGC | |
| | CAGGAGGACGCUCCAGAUGAAAUGGGUAAGUACAUCAAGAG | |
| | CUUCGUGGAGCGCGUGCUGAAGAACGAGCAGUAA | |

### Example 5

Except for the change in the target RNA from GFP_A200 to Rluc_W104X. (SEQ ID NO: 45), ADg_Rluc (SEQ ID NO: 44) having the same base sequence as the editing guide RNA of Reference Example 2 and cpADg_Rluc having the same base sequence as the editing guide RNA of Example 2 were designed, and plasmids expressing these were constructed by a conventional method. In addition, racADg_Rluc (SEQ ID NO: 46) was designed by adding a nucleotide sequence that enables intracellular circularization reaction to the nucleotide sequence of cpADg_Rluc, and a plasmid expressing this was constructed.

TABLE 14

| | | |
|---|---|---|
| ADg_Rluc | GAAGGUUCAGCAGCUCGAACCAAGGGGUGGAAUAGUAUA | SEQ ID NO: 44 |
| | ACAAUAUGCUAAAUGUUGUUAUAGUAUCCCACCUU | |
| cpADg_Rluc | AAAUGUUGUUAUAGUAUCCCACCUCGACGUUGUAAAACG | SEQ ID NO: 45 |
| | ACGGCCAGUGAAUUCGAAGGUUCAGCAGCUCGAACCAAG | |
| | GGGUGGAAUAGUAUAACAAUAUGCUU | |
| racADg_Rluc | GCCAUCAGUCGCCGGUCCCAAGCCCGGAUAAAAUGGGAG | SEQ ID NO: 46 |
| | GGGGGGGGAAACCGCCUAACCAUGCCGACUGAUGGCAGG | |
| | AGACGGUCGGGUCCACGAAAUGUUGUUAUAGUAUCCCAC | |
| | CUCGACGUUGUAAAACGACGGCCAGUGAAUUCGAAGGUU | |
| | CAGCAGCUCGAACCAAGGGGUGGAAUAGUAUAACAAUAU | |
| | GCUCGUGUCGAGUAGAGUGUGGGCUCCUGCCAUCAGUCG | |
| | GCGUGGACUGUAGAACACUGCCAAUGCCGGUCCCAAGCC | |
| | CGGAUAAAAGUGGAGGGUACAGUCCACGCUU | |
| circADg_Rluc | AACCAUGCCGACUGAUGGCAGGAGACGGUCGGGUCCACG | SEQ ID NO: 56 |
| | AAAUGUUGUUAUAGUAUCCCACCUCGACGUUGUAAAACG | |
| | ACGGCCAGUGAAUUCGAAGGUUCAGCAGCUCGAACCAAG | |
| | GGGUGGAAUAGUAUAACAAUAUGCUCGUGUCGAGUAGAG | |
| | UGUGGGCUCCUGCCAUCAGUCGGCGUGGACUGUAG | |

In the nucleotide sequence of racADg_Rluc, the underlined portion indicates the ribozyme region (rib sequence) and the double underlined portion indicates the ligation stem region. The nucleotide sequence of racADg_Rluc contains a Broccoli sequence between the ligation stem region and cpADg_Rluc. Shown below are the schematic structures of ADg_Rluc, cpADg_Rluc, and circADg_Rluc, which is after cyclization of racADg_Rluc.

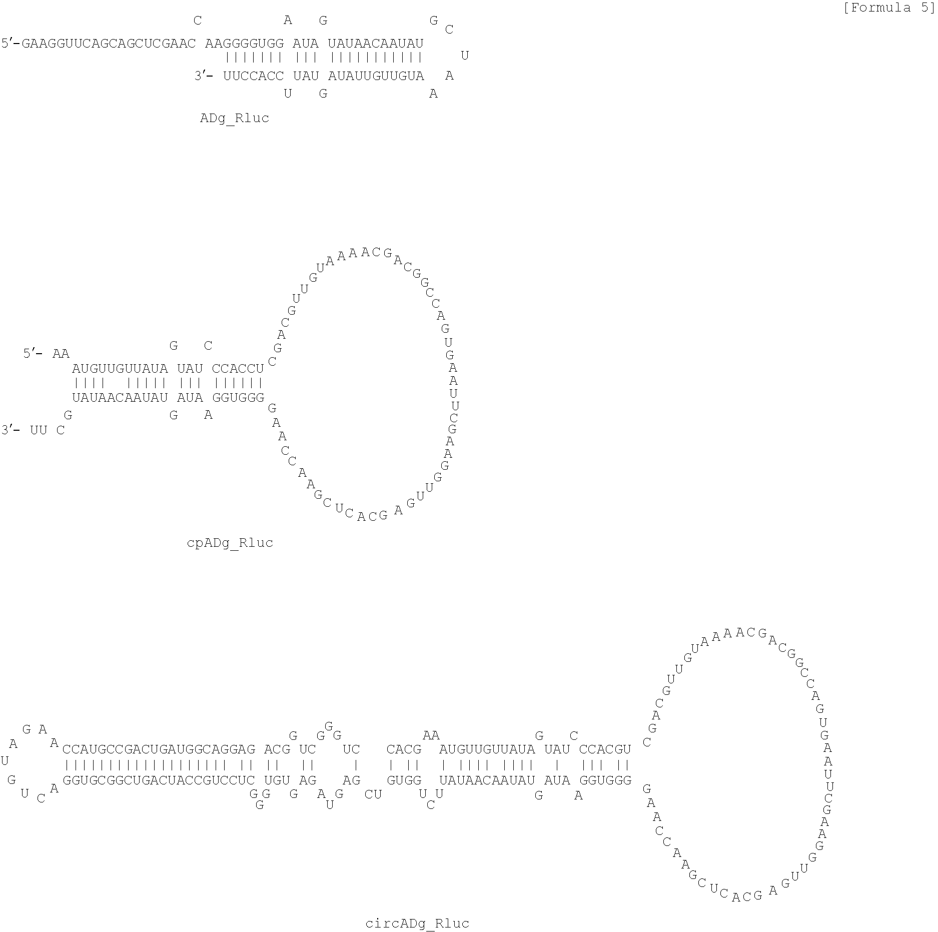

### Cell Culture

HEK293 cells were seeded on a 24-well plate at 5.0×104 cells/well and cultured for 48 hours. Using X-tremeGENE (TM) HP DNA Transfection Reagent (manufactured by Roche), 50 ng of the Rluc_W104X expression plasmid, 250 ng of the plasmid expressing the target editing guide RNA, and 250 ng of the ADAR expression plasmid were transfected and cultured for 72 hours. As ADAR expression plasmids, a plasmid expressing ADAR2, a plasmid expressing ADAR1p110, and a plasmid expressing ADAR1p150 were used.

### Editing Analysis

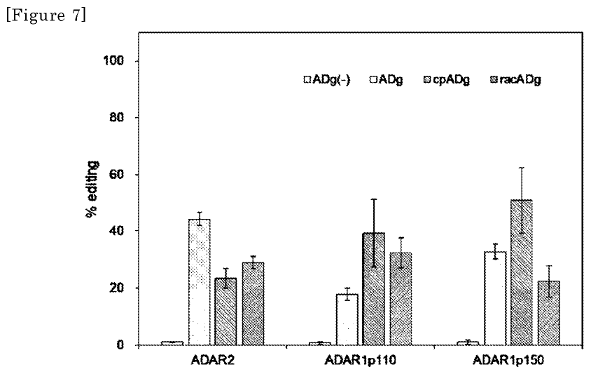

Total RNA was extracted from the cells cultured in a 24-well plate using Sepasol RNA I Super G (manufactured by Nacalai Tesque Inc.) and subjected to DNase treatment using Recombinant DNase I (manufactured by Takara Holdings inc.). The recovered RNA sample was subjected to a reverse transcription reaction using PrimeScript Reverse Transcriptase II (manufactured by Takara Bio Inc.) to synthesize cDNA. Using the obtained cDNA as a template, 1st PCR was performed using Prime Star GXL (manufactured by Takara Bio Inc.), 0.3 UM of the Rluc_full_F01 primer (SEQ ID NO: 48), and 0.3 μM of the 3′-Adp primer (SEQ ID NO: 49). The Rluc fragment was amplified by performing the 2nd PCR using cDNA obtained by diluting the 1st PCR product 200 fold as a template, Prime Star GXL (manufactured by Takara Bio), 0.3 μM Rluc_2 ndPCR_F01 (SEQ ID NO: 50), and 0.3 μM Rluc_A311_s01_R01 (SEQ ID NO: 51). Direct sequencing of the dsDNA amplified with 0.165 μM of the Rluc_A311_seqF01 primer (SEQ ID NO: 52) was performed using the Big Dye Terminator v3.1 Cycle Sequencing Kit. Finally, the editing ratio (%) was calculated from the peak height ratio G/(G+A) of the obtained chromatographic chart. The results are shown in Table 16 and FIG. 7.

TABLE 15

| | | |
|---|---|---|
| Oligo (dT) 17 | GGCCACGCGTCGACTAGTACTTTTTTTTTTTTTTTTT | SEQ ID NO: 47 |
| Rluc_full_F01 | CATGGCTTCCAAGGTGTACG | SEQ ID NO: 48 |
| 3′-Adp | GGCCACGCGTCGACTAGTAC | SEQ ID NO: 49 |
| Rluc_2ndPCR_F01 | GCATGATCACTGGGCCTCAG | SEQ ID NO: 50 |
| Rluc_A311_01_R01 | CCAGTCGTGGCCCACAAAG | SEQ ID NO: 51 |
| Rluc_A311_seqF01 | GCTGGACTCCTTCATCAAC | SEQ ID NO: 52 |

### Luciferase Reporter Assay

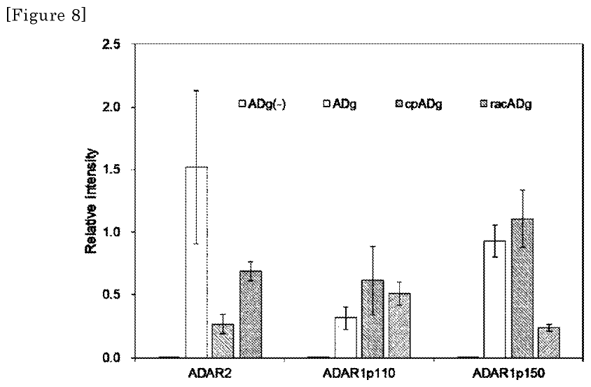

The Dual-Luciferase Reporter Assay System (manufactured by Promega Corporation) was used. A cell extract was obtained using 100 μL of the Passive Lysis Buffer on the cells cultured in a 24-well plate. 100 μL of LARII was added to 20 μL of the obtained cell extract, and after 60 seconds, the luminescence intensity of Fluc was measured with the GloMax® 20/20 Luminometer (manufactured by Promega Corporation). After that, 100 μL of the Stop & Glo Reagent was added, and the luminescence intensity of Rluc was measured 60 seconds later. The luminescence intensity was normalized by Fluc. The results are shown in Table 16 and FIG. 8.

TABLE 16

| | | | | Relative luminescence | |
|---|---|---|---|---|---|
| | | Editing Ratio (%) | | intensity | |
| | | Mean | S.D. | Mean | S.D. |
| ADAR | gRNA | (n = 3) | (n = 3) | (n = 3) | (n = 3) |
| — | — | 0.5 | 0.2 | 0.000 | 0.000 |
| ADAR2 | — | 1.0 | 0.3 | 0.002 | 0.000 |
| | ADg | 44.3 | 2.4 | 1.519 | 0.613 |
| | cpADg | 23.6 | 3.4 | 0.269 | 0.077 |
| | racADg | 29.1 | 2.1 | 0.688 | 0.079 |
| ADAR1 p110 | — | 0.8 | 0.4 | 0.000 | 0.000 |
| | ADg | 17.8 | 2.1 | 0.315 | 0.087 |
| | cpADg | 39.3 | 12.0 | 0.615 | 0.272 |
| | racADg | 32.5 | 5.2 | 0.510 | 0.095 |
| ADAR1p150 | — | 1.1 | 0.8 | 0.001 | 0.000 |
| | ADg | 32.8 | 2.7 | 0.927 | 0.128 |
| | cpADg | 50.8 | 11.5 | 1.105 | 0.227 |
| | racADg | 22.4 | 5.5 | 0.239 | 0.029 |

cpADg is less efficient than conventional ADg in the induction of ADAR2 editing, but has a higher efficiency in the induction of ADAR1 editing. This result indicates that ADg and cpADg have different ADAR selectivities. In addition, racADg is considered to induce target editing as a circular RNA in which the rib sequence is excised in the cell. Since racADg is in the form of cpADg added with a sequence for intracellular circularization, its editing-inducing activity is expected to be equal to or lower than that of cpADg. Nevertheless, the editing induction results for ADAR2 in cell assays show higher values for circularized RNA than for cpADg. This can be interpreted as indication of a positive effect of circularization, that is, improved stability in the intracellular environment.

### Confirmation of the Circular RNA Expression

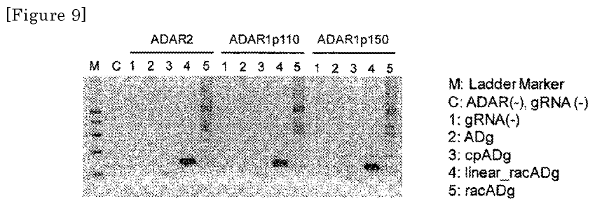

RNA samples collected from the cultured cells were subjected to a reverse transcription reaction using PrimeScript Reverse Transcriptase II (manufactured by Takara Bio Inc.) and 2.5 μM of the racADg_RT_L30_R01 primer to synthesize cDNA. Using the resulting cDNA as a template, PCR was performed using Prime Star GXL (manufactured by Takara Bio Inc.), 0.3 μM of the racADg_RT_L30_F01 primer (SEQ ID NO: 53), and 0.3 μM of the racADg_RT_L30_R01 primer (SEQ ID NO: 54). After that, electrophoresis was performed using a 2.0% agarose gel. The results are shown in FIG. 9. Further, linear_racADg_Rluc (SEQ ID NO: 55) is a target editing guide RNA that inactivates the ribozyme region of racADg_Rluc.

TABLE 17

| | | |
|---|---|---|
| racADg_RT_L30_F01 | CCTCGACGTTGTAAAACG | SEQ ID NO: 53 |
| racADg_RT_L30_R01 | CATGGTTCTACAGTCCAC | SEQ ID NO: 54 |
| linear_racADg_Rluc | GGGGGGGGGAAACCGCCUAACCAUGCCGACUGAUGGC | SEQ ID NO: 55 |
| | AGGAGACGGUCGGGUCCACGAAAUGUUGUUAUAGUAU | |
| | CCCACCUCGACGUUGUAAAACGACGGCCAGUGAAUUC | |
| | GAAGGUUCAGCAGCUCGAACCAAGGGGUGGAAUAGUA | |
| | UAACAAUAUGCUCGUGUCGAGUAGAGUGUGGGCUCCU | |
| | GCCAUCAGUGGGCGUGGACUGUAGAACACUGCCAAUG | |
| | CUU | |

As shown in FIG. 9, linear_racADg_Rluc yielded a single amplification product, whereas racADg_Rluc yielded multiple amplification products with different molecular weights. This indicates that racADg_Rluc is circularized in cells. When a reverse transcription reaction is carried out using a circular RNA as a template under general conditions, the reverse transcription elongation reaction does not terminate due to the lack of a terminus, resulting in a cDNA having repeated sequences called concatemers. Therefore, in principle, when RT-PCR is performed on a normal single-stranded RNA template, there will be only one amplified product, but when cDNA obtained with the circular RNA as a template is used as a template, in addition to the amplified product obtained as a linear strand, multiple products are obtained by lengthening as a result of repeating and adding units of a certain length. That is, whether the template cDNA is linear or circular can be determined by analyzing the pattern of the RT-PCR amplification products. From the above, it was shown that the prepared racADg_Rluc is circularized in cells.

### Example 6

In the same manner as above, a plasmid expressing a target editing guide RNA with the schematic structure shown below was constructed. Further, the editing target RNA is GFP A200.

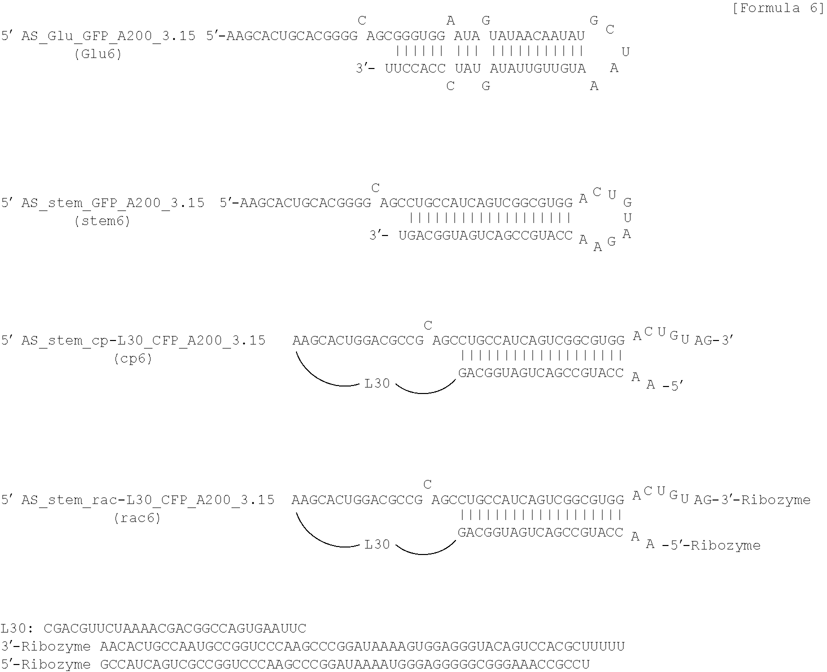

TABLE 18

| | | |
|---|---|---|
| 5′ AS_Glu_GFP_A200_3 15 | AAGCACUGCACGCCGCAGGGGGUGGAAUAGUAUAACAAUA | SEQ ID NO: 57 |
| (Glu6) | UGCUAAAUGUUGUUAUAGUAUCCCACCU | |
| 5′ AS_stem_GFP_A200_3.15 | AAGCACUGCACGCCGCAGCCUGCCAUCAGUCGGCGUGGAC | SEQ ID NO: 58 |
| (sten6) | UGUAGAACCAUGCCGACUGAUGGCAG | |
| 5′ AS_sten_cp-L30_GFP_A200_3.15 | AACCAUGCCGACUGAUGGCAGCGACGUUGUAAAACGACGG | SEQ ID NO: 59 |
| (cp6) | CCAGUGAAUUCAAGCACUGCACGCCGCAGCCUGCCAUCAG | |
| | UCGGCGUGGACUGUAG | |
| 5′ AS_stem_rac-L30_GFP_A200_3.15 | GCCAUCAGUCGCCGGUCCCAAGCCCGGAUAAAAUGGGAGG | SEQ ID NO: 60 |
| (rac6) | GGGCGGGAAACCGCCUAACCAUGCCGACUGAUGGCAGCGA | |
| | CGUUGUAAAACGACGGCCAGUGAAUUCAAGCACUGCACGC | |
| | CGCAGCCUGCCAUCAGUCGGCGUGGACUGUAGAACACUGC | |
| | CAAUGCCGGUCCCAAGCCCGGAUAAAAGUGGAGGGUACAG | |
| | UCCACGCUUUUU | |
| 5′ AS_stem_circ-L30_GFP_A200_3.15 | AACCAUGCCGACUGAUGGCAGCGACGUUGUAAAACGACGG | SEQ ID NO: 61 |
| (circ6) | CCAGUGAAUUCAAGCAGUGCACGCCGCAGCCUGCCAUCAG | |
| | UCGGCGUGGACUGUAG | |

Of these, 5′AS_stem_rac-L30_GFP_A200_3.1 5 (rac6) is thought to form a target-editing guide RNA (circ6) of the schematic structure below through progression of an intracellular circularization reaction by ribozyme.

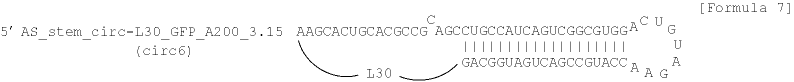

### Cell Culture

HEK293 cells were seeded on a 24-well plate at 5.0×10⁴ cells/well and cultured for 48 hours. Using Lipofectamine® 3000 Reagent (Thermo Fisher Scientific), 10 ng of the GFP A200 expression plasmid, 250 ng of a plasmid expressing the target editing guide RNA, and 250 ng of the ADAR expression plasmid were transfected and cultured for 48 hours. A plasmid expressing ADAR2 was used as the ADAR expression plasmid.

### Editing Analysis

Total RNA was extracted from the cells cultured in a 24-well plate using Sepasol RNA I Super G (manufactured by Nacalai Tesque Inc.) and subjected to DNase treatment using Recombinant DNase I (manufactured by TaKaRa). The recovered RNA sample was subjected to a reverse transcription reaction using PrimeScript Reverse Transcriptase II (manufactured by Takara Bio Inc.) to synthesize cDNA.

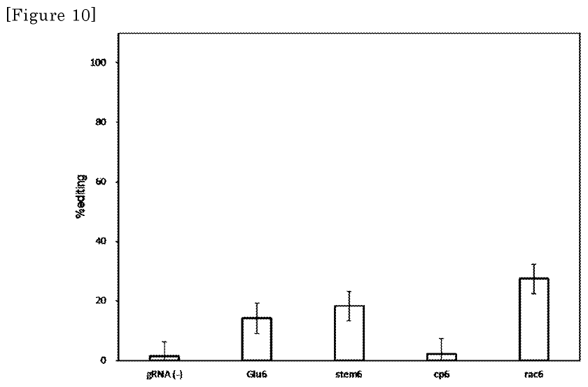

Using the resulting cDNA as a template, dsDNA was amplified by PCR using 0.3 M of the T7GFP_sRNA_F01 primer (SEQ ID NO: 40) and 0.3 UM of the GFP_sRNA_R01 primer (SEQ ID NO: 41). Direct sequencing of the dsDNA amplified with 0.165 μM of the T7proGGG primer (SEQ ID NO: 42) was performed using the Big Dye Terminator v3.1 Cycle Sequencing Kit. Finally, the editing ratio (%) was calculated from the peak height ratio G/(G+A) in the obtained chromatographic chart. The results are shown in Table 19 and FIG. 10.

TABLE 19

Editing Ratio (%)

| gRNA | Mean (n = 3) | S.D. (n = 3) |
|---|---|---|
| — | 1.4 | 0.2 |
| Glu6 | 14.1 | 1.4 |
| stem6 | 18.2 | 0.6 |
| cp6 | 2.3 | 0.8 |
| rac6 | 27.3 | 1.2 |

rac6, which generates a circular-type target editing guide RNA, has higher intracellular editing induction efficiency than the conventional type Glu6 and stem6.

### Example 7

In the same manner as above, a plasmid expressing a target editing guide RNA with the schematic structure shown below was constructed. Further, the editing target RNA is GFP A173. GFP A173 was obtained by converting 58W (tryptophan) in the GFP encoding region to 58X (termination codon). Specifically, the editing target was set by mutating 173G, which corresponds to the 58th tryptophan, to 173A.

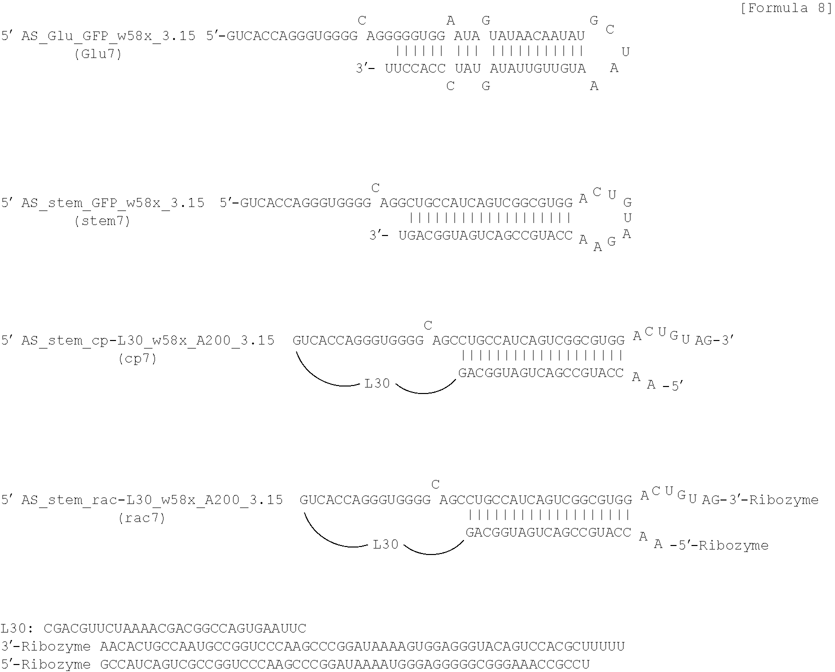

TABLE 20

| | | |
|---|---|---|
| 5′ AS_Glu_GFP_w58x_3.15 | GUCACCAGGGUGGGCCAGGGGGUGGAAUAGUAUAACAAUA | SEQ ID NO: 62 |
| (Glu7) | UGCUAAAUGUUGUUAUAGUAUCCCACCU | |
| 5′ AS_stem_GFP_w58x_3.15 | GUCACCAGGGUGGGCCAGGCUGCCAUCAGUCGGCGUGGAC | SEQ ID NO: 63 |
| (stem7) | UGUAGAACCAUGCCGAGUGAUGGCAG | |
| 5′ AS_stem_cp-L30_GFP_w58x_3.15 | AACCAUGCCGACUGAUGGCAGCGACGUUGUAAAACGACGG | SEQ ID NO: 64 |
| (cp7) | CCAGUGAAUUCAAGCACUGCACGCCGCAGCCUGCCAUCAG | |
| | UCGGCGUGGACUGUAG | |
| 5′ AS_stem_rac-L30_GFP_w58x_3.15 | GCCAUCAGUCGCCGGUCCCAAGCCCGGAUAAAAUGGGAGG | SEQ ID NO: 65 |
| (rac7) | GGGCGGGAAACCGCCUAACCAUGCCGACUGAUGGCAGCGA | |
| | CGUUGUAAAACGACGGCCAGUGAAUUCGUCACCAGGGUGG | |
| | GCCAGGCUGCCAUCAGUCGGCGUGGACUGUAGAACACUGC | |
| | CAAUGCCGGUCCCAAGCCCGGAUAAAAGUGGAGGGUACAG | |
| | UCCACGCUUUUU | |
| 5′ AS_stem_circ-L30_GFP_w58x_3.15 | AACCAUGCCGACUGAUGGCAGCGACGUUGUAAAACGACGG | SEQ ID NO: 66 |
| (circ7) | CCAGUGAAUUCGUCACCAGGGUGGGCCAGGCUGCCAUCAG | |
| | UGGGCGUGGACUGUAG | |

Of these, 5′AS_stem_rac-L30_GFP_w58x_3.15 (rac7) is thought to form a target-editing guide RNA (circ7) of the schematic structure below through progression of an intracellular circularization reaction by ribozyme.

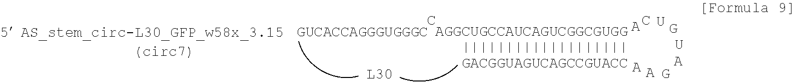

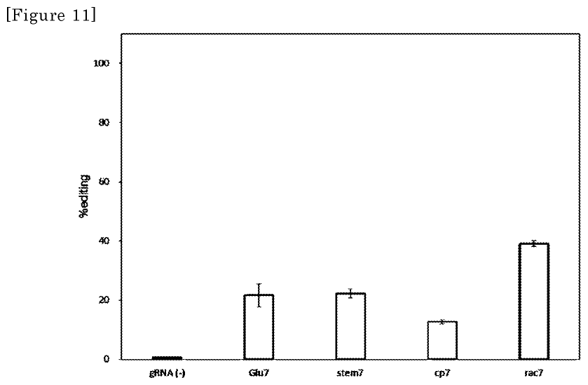

Cell culture and editing analysis were performed in the same manner as in Example 6. Results are shown in Table 21 and FIG. 11.

TABLE 21

Editing Ratio (%)

| gRNA | Mean (n = 3) | S.D. (n = 3) |
|---|---|---|
| — | 1.4 | 0.2 |
| Glu7 | 14.1 | 1.4 |
| stem7 | 18.2 | 0.6 |
| cp7 | 2.3 | 0.8 |
| rac7 | 27.3 | 1.2 |

Even when the editing target RNA is replaced, rac7, which produces a circular type target editing guide RNA, has higher intracellular editing induction efficiency than the conventional type Glu7 and stem7.

### Example 8

In the same manner as above, a plasmid expressing a target editing guide RNA with the schematic structure shown below was constructed. The editing target RNA is Rluc_W104X (Rluc_A311) constructed in Reference Example 3.

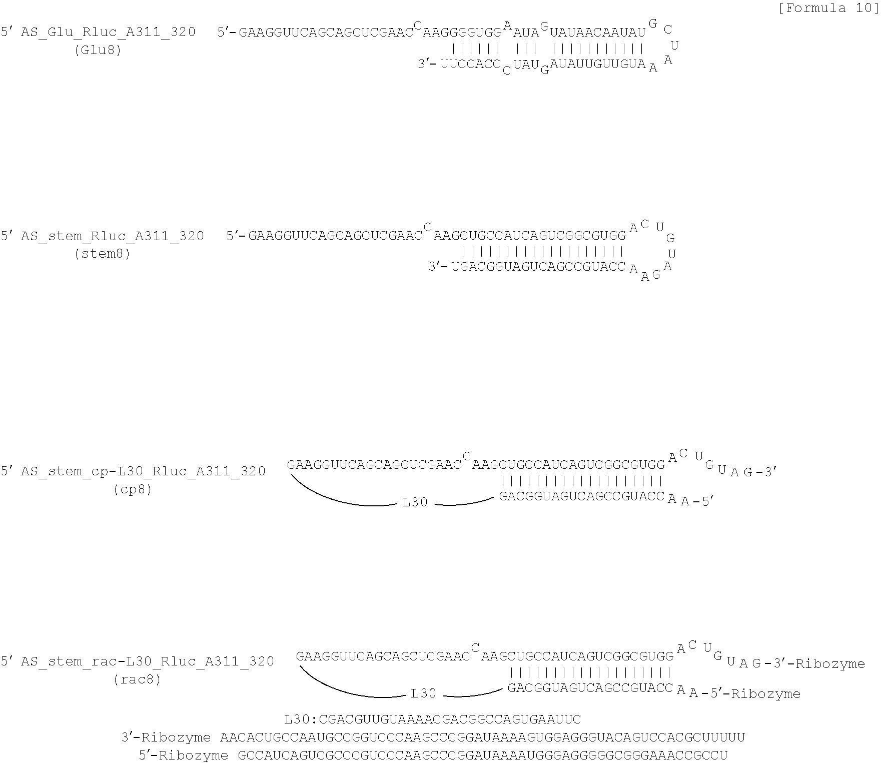

TABLE 22

| | | |
|---|---|---|
| 5′ AS_Glu_Rluc_A311_3.20 | GAAGGUUCAGCAGCUCGAACCAAGGGGUGGAAUAGUAUAA | SEQ ID NO: 67 |
| (Glu8) | CAAUAUGCUAAAUGUUGUUAUAGUAUCCCACCU | |
| 5′ AS_sten_Rluc_A311_3.20 | GAAGGUUCAGCAGCUCGAACCAAGCUGCCAUCAGUCGGCG | SEQ ID NO: 68 |
| (stem8) | UGGACUGUAGAACCAUGCCGACUGAUGGCAG | |
| 5′ AS_stem_cp-L30_Rluc_A311_3.20 | AACCAUGCCGACUGAUGGCAGCGACGUUGUAAAACGACGG | SEQ ID NO: 69 |
| (cp8) | CCAGUGAAUUCGAAGGUUCAGCAGCUCGAACCAAGCUGCC | |
| | AUCAGUCGGCGUGGACUGUAG | |
| 5′ AS_stem_rac-L30_Rluc_A311_3.20 | GCCAUCAGUCGCCGGUCCCAAGCCCGGAUAAAAUGGGAGG | SEQ ID NO: 70 |
| (rac8) | GGGGGGGAAACCGCCUAACCAUGCCGACUGAUGGCAGCGA | |
| | CGUUGUAAAACGACGGCCAGUGAAUUCGAAGGUUCAGCAG | |
| | CUCGAACCAAGCUGCCAUGAGUCGGCGUGGACUGUAGAAG | |
| | ACUGCCAAUGCCGGUCCCAAGCCCGGAUAAAAGUGGAGGG | |
| | UACAGUCCACGCUUUUU | |
| 5′ AS_stem_circ-L30_Rluc_A311_3.20 | AACCAUGCCGACUGAUGGCAGCGACGUUGUAAAACGACGG | SEQ ID NO: 71 |
| (circ8) | CCAGUGAAUUCGAAGGUUCAGCAGCUCGAACCAAGCUGCC | |
| | AUCAGUCGGCGUGGACUGUAG | |

Of these, 5′AS_stem_rac-L30_Rluc_A311_3.20 (rac8) is thought to form a target-editing guide RNA (circ8) of the schematic structure below through progression of an intracellular circularization reaction by ribozyme.

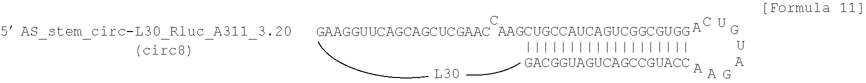

### Cell Culture

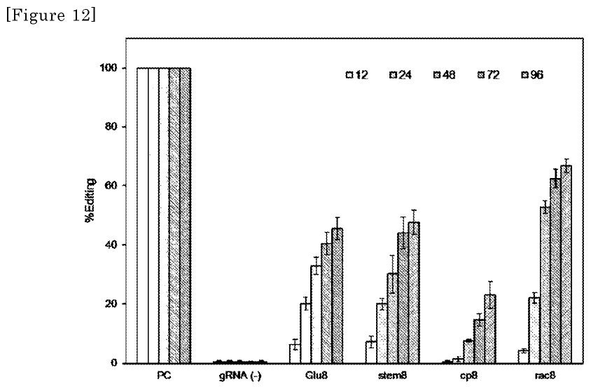

HEK293 cells were seeded on a 24-well plate at 5.0×10⁴ cells/well and cultured for 48 hours. Using Lipofectamine® 3000 Reagent (Thermo Fisher Scientific), 10 ng of the Rluc A311 expression plasmid, 250 ng of a plasmid expressing the target editing guide RNA, and 250 ng of the ADAR expression plasmid were transfected. A plasmid expressing ADAR2 was used as the ADAR expression plasmid. The incubation time after transfection was 12 hours, 24 hours, 48 hours, 7 2 hours or 96 hours. Editing analysis and luciferase reporter assays were performed at each incubation time as in the editing analysis and luciferase reporter assay of Example 5, and the changes in editing ratio (%) and relative luminescence intensity over time were measured. The results of the editing analysis are shown in Table 23 and FIG. 12, and the results of the luciferase reporter assay are shown in Table 24 and FIG. 1 3. Note that PC is the Positive Control and the results were obtained using a reporter expression plasmid whose target site is the natural form of G.

TABLE 23

| | Incubation Time (hrs) | | | | |
|---|---|---|---|---|---|
| gRNA | 12 | 24 | 48 | 72 | 96 |
| PC | 100.0 | 100.0 | 100.0 | 100.0 | 100.0 |
| — | 0.5 | 0.6 | 0.6 | 0.6 | 0.6 |
| Glu8 | 6.2 | 20.0 | 32.9 | 40.5 | 45.4 |
| stem8 | 7.2 | 20.0 | 30.0 | 44.2 | 47.6 |
| cp8 | 0.5 | 1.5 | 7.6 | 14.6 | 23.0 |
| rac8 | 4.2 | 22.0 | 52.7 | 62.4 | 66.8 |

% editing

TABLE 24

| | Incubation Time (hrs) | | | | |
|---|---|---|---|---|---|
| gRNA | 12 | 24 | 48 | 72 | 96 |
| PC | 8.0 | 10.2 | 11.7 | 16.2 | 13.0 |
| — | 0.0 | 0.0 | 0.0 | 0.0 | 0.0 |
| Glu8 | 0.0 | 0.1 | 0.5 | 0.8 | 1.1 |
| stem8 | 0.0 | 0.1 | 0.4 | 0.9 | 1.1 |
| cp8 | 0.0 | 0.0 | 0.1 | 0.1 | 0.3 |
| rac8 | 0.0 | 0.3 | 1.6 | 3.3 | 4.1 |

As for the editing ratio, rac8, which produces a circular target editing guide RNA, has higher intracellular editing induction efficiency than the conventional type Glu8 and stem8 after 24 hours of culturing or longer. Also, at the protein expression level, rac8, which produces circular target editing guide RNAs, is clearly superior to the conventional type Glu8 and stem8.

### Example 9

### Cell Culture

HeLa cells were seeded on a 24-well plate at 5.0×10⁴ cells/well and cultured for 48 hours. Using Lipofectamine® 3000 Reagent (Thermo Fisher Scientific), 50 ng of the Rluc A311 expression plasmid and 500 ng of a plasmid expressing the target editing guide RNA were transfected and cultured for 72 hours. Further, stem8, cp8 and rac8 were used as plasmids expressing the target editing guide RNA, and no ADAR expression plasmid was used.

Proteins were collected from the cultured cells and confirmed by Western blotting to express ADAR1p150, ADAR1p110 and ADAR2.

### Luciferase Reporter Assay

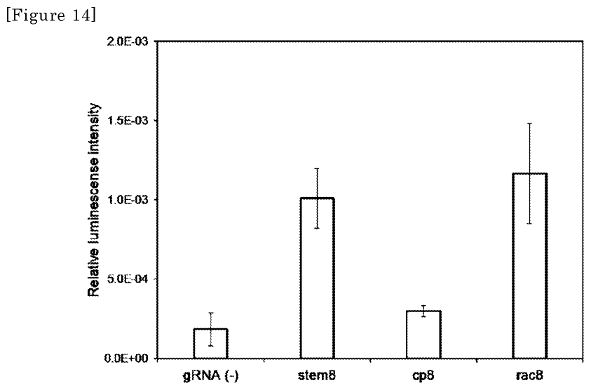

The editing-inducing activity by endogenous ADAR was evaluated in the same manner as the luciferase reporter assay in Example 5. Results are shown in Table 25 and FIG. 14.

TABLE 25

Relative luminescence intensity

| gRNA | Mean (n = 3) | S.D. (n = 3) |
|---|---|---|
| — | 1.85E−04 | 1.04E−04 |
| stem8 | 1.01E−03 | 1.88E−04 |
| cp8 | 2.98E−04 | 3.46E−05 |
| rac8 | 1.17E−03 | 3.17E−04 |

Target editing guide RNAs were shown to be able to induce editing activity of endogenous ADARs.

### Example 10

Target editing guide RNAs with different lengths and sequences of the first linker (Linker) whose schematic structure is shown below were synthesized by in vitro transcription in the same manner as above. The editing target RNA is GFP A200.

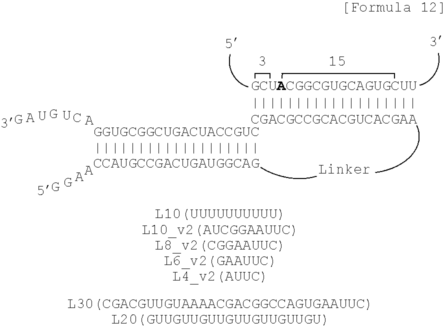

TABLE 26

| | | |
|---|---|---|
| 5′ AS_stem_cp-L10_GFP_A200 | GGAACCAUGCCGACUGAUGGCAGUUUUUUUUUUAAGCACU | SEQ ID NO: 72 |
| (L10) | GCACGCCGCAGCCUGCCAUCAGUCGGCGUGGACUGUAG | |
| 5′ AS_stem_cp-L10v_GFP_A200 | GGAACCAUGCCGACUGAUGGCAGAUCGGAAUUCAAGCACU | SEQ ID NO: 73 |
| (10v) | GCACGCCGCAGCCUGCGAUCAGUCGGCGUGGACUGUAG | |
| 5′ AS stem cp-L8v_GFP_A200 | GGAACCAUUCCGACUGAUGGCAGCGGAAUUCAAGCACUGC | SEQ ID NO: 74 |
| (18v) | ACGCCGCAGCCUGCCAUCAGUCGGCGUGGACUGUAG | |
| 5′ AS_stem_cp-L6v_GFP_A200 | GGAACGAUGCCGACUGAUGGCAGGAAUUCAAGCACUGCAC | SEQ ID NO: 75 |
| (LGv) | GCCGCAGCCUGCCAUCAGUCGGCGUGGACUGUAG | |
| 5′ AS_stem_cp-L4v_GFP_A200 | GGAACCAUGCCGACUGAUGGCAGAUUCAAGCAGUGCACGC | SEQ ID NO: 76 |
| (L4v) | CGCAGCCUGCCAUCAGUCGGCGUGGACUGUAG | |
| 5′ AS_stem_cp-L30_GFP_A200 | GGAACCAUGCCGACUGAUGGCAGCGACGUUGUAAAACGAC | SEQ ID NO: 77 |
| (L30) | GGCCAGUGAAUUCAAGCACUGCACGCCGCAGCCUGCCAUC | |
| | AGUCGGCGUGGACUGUAG | |
| 6′ AS_stem_cp-L20_GFP_A200 | GGAACCAUGCCGACUGAUGGCAGGUUGUUGUUGUUGUUGU | SEQ ID NO: 78 |
| (L20) | UGUAAGCACUGCACGCCGCAGCCUGCCAUCAGUCGGCGUG | |
| | GACUGUAG | |
| target | GCUACGGCGUGCAGUGCUU | SEQ ID NO: 79 |

### Editing Analysis

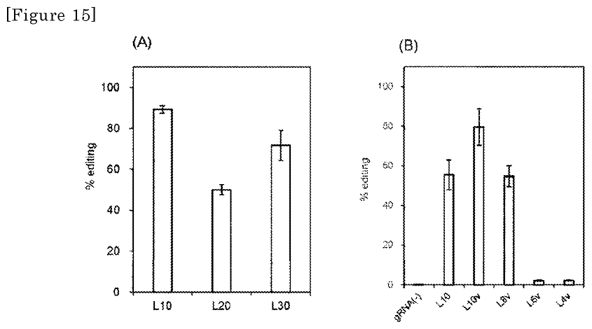

The editing-inducing activity of the target-editing guide RNA was evaluated in the same manner as in Evaluation 1 above. Results are shown in Table 27 and FIG. 15. FIG. 15(A) is a figure showing editing-inducing activity when the length of the first linkage group is 10 nt, 20 nt, or 30 nt. FIG. 15(B) is a figure showing the editing-inducing activity when the length of the first linkage group is from 4 nt to 10 nt.

TABLE 27

Editing Ratio (%)

| gRNA | Mean (n = 3) | S.D. (n = 3) |
|---|---|---|
| L10 | 89.3 | 1.9 |
| L20 | 50.0 | 2.5 |
| L30 | 71.7 | 7.4 |

TABLE 28

Editing Ratio (%)

| gRNA | Mean (n = 3) | S.D. (n = 3) |
|---|---|---|
| — | 0.3 | 0.2 |
| L10 | 55.4 | 7.5 |
| L10v | 79.6 | 9.2 |
| L8v | 54.8 | 5.4 |
| L6v | 2.1 | 0.3 |
| L4v | 2.1 | 0.3 |

It was suggested that when the editing target RNA is GFP A200, the appropriate length of the first linkage group is about 8 nt to 10 nt.

The disclosure of Japanese Patent Application No. 2020-043253 (Filing Date: Mar. 12, 2020) is incorporated herein by reference in its entirety. All documents, patent applications, and technical standards described herein are incorporated herein by reference to the same extent as if the individual documents, patent applications, and technical standards were specifically and individually stated to be incorporated by reference.

1. An oligonucleotide that induces site-specific editing of a target RNA, comprising:
a first oligonucleotide that identifies the target RNA,
a second oligonucleotide linked to the 3′ side of the first oligonucleotide,
a third oligonucleotide having a base sequence capable of forming a complementary strand with the second oligonucleotide, and
a first linkage group that links the 5′ end of the first oligonucleotide and the 3′ end of the third oligonucleotide,
wherein the first oligonucleotide consists of:
a target-corresponding nucleotide residue that corresponds to an adenosine residue in the target RNA,
a 10 to 24 residue oligonucleotide linked to the 5′ side of the target-corresponding nucleotide residue and having a base sequence complementary to the target RNA, and
a 2 to 7 residue oligonucleotide linked to the 3′ side of the target-corresponding nucleotide residue and having a base sequence complementary to the target RNA.

2. The oligonucleotide of claim 1, further comprising a second linkage group that links the 3′ end of the second oligonucleotide to the 5′ end of the third oligonucleotide.

3. The oligonucleotide of claim 2, wherein the second linkage group comprises a nucleotide chain of 4 to 20 residues.

4. The oligonucleotide of claim 1, wherein a number of residues of the second oligonucleotide is 2 or more and 30 or less.

5. The oligonucleotide of claim 1, wherein the first linkage group comprises a nucleotide chain of 8 to 50 residues.

6. A method for site-specific editing of a target RNA, comprising contacting the oligonucleotide of claim 1 with the target RNA in the presence of adenosine deaminase.

7. The editing method of claim 6, wherein the method is performed in eukaryotic cells.


SEQUENCE LISTING (79 sequences)

SEQ ID NO: 1 - RNA, 22 nt, Artificial Sequence
  ARR
ggguggaauaguauaacaauau

SEQ ID NO: 2 - RNA, 22 nt, Artificial Sequence
  ARR
auguuguuauaguaucccaccu

SEQ ID NO: 3 - RNA, 25 nt, Artificial Sequence
  ARR
ggguggaauaguauaacaauaugcu

SEQ ID NO: 4 - RNA, 26 nt, Artificial Sequence
  ARR
ggaaauguuguuauaguaucccaccu

SEQ ID NO: 5 - RNA, 26 nt, Artificial Sequence
  ARR
cugccaucagucggcguggacuguag

SEQ ID NO: 6 - RNA, 23 nt, Artificial Sequence
  ARR
ggaaccaugccgacugauggcag

SEQ ID NO: 7 - RNA, 10 nt, Artificial Sequence
  ARR
ggguggaaua

SEQ ID NO: 8 - RNA, 10 nt, Artificial Sequence
  ARR
uaucccaccu

SEQ ID NO: 9 - RNA, 56 nt, Artificial Sequence
  ribozyme
gccaucagucgccggucccaagcccggauaaaaugggagggggcgggaaaccgccu

SEQ ID NO: 10 - RNA, 19 nt, Artificial Sequence
  LgSTEM
ccaugccgacugauggcag

SEQ ID NO: 11 - RNA, 19 nt, Artificial Sequence
  LgSTEM
cugccaucagucggcgugg

SEQ ID NO: 12 - RNA, 55 nt, Artificial Sequence
  ribozyme
aacacugccaaugccggucccaagcccggauaaaaguggaggguacaguccacgc

SEQ ID NO: 13 - RNA, 49 nt, Artificial Sequence
  ARR
ggguggaauaguauaacaauaugcuaaauguuguuauaguaucccaccu

SEQ ID NO: 14 - RNA, 47 nt, Artificial Sequence
  ARR
cugccaucagucggcguggacuguagaaccaugccgacugauggcag

SEQ ID NO: 15 - RNA, 49 nt, Artificial Sequence
  ARR
cugccaucagucggcguggacuguagggaaccaugccgacugauggcag

SEQ ID NO: 16 - RNA, 24 nt, Artificial Sequence
  ARR
ggguggaauauucguaucccaccu

SEQ ID NO: 17 - RNA, 16 nt, Artificial Sequence
  ARR
gggugguucgccaccu

SEQ ID NO: 18 - RNA, 14 nt, Artificial Sequence
  ARR
ggguguucgcaccu

SEQ ID NO: 19 - RNA, 12 nt, Artificial Sequence
  ARR
ggguuucgaccu

SEQ ID NO: 20 - DNA, 40 bp, Artificial Sequence
  F-primer
ctaatacgactcactatagggtgaatggccacaagttcag

SEQ ID NO: 21 - DNA, 20 bp, Artificial Sequence
  R-primer
tagcgtgagaagcactgcac

SEQ ID NO: 22 - RNA, 160 nt, Artificial Sequence
  modified GFP
gggugaauggccacaaguucagcgugagcggcgagggcgagggcgaugccaccuacggcaagcugacccugaaguucaucugcaccaccggcaagcugccugugcccuggcccacccuggugaccacccugagcuacggcgugcagugcuucucacgcua

SEQ ID NO: 23 - RNA, 70 nt, Artificial Sequence
  gRNA
ggaagcacugcacgccgcagcggguggaauaguauaacaauaugcuaaauguuguuauaguaucccaccu

SEQ ID NO: 24 - DNA, 63 bp, Artificial Sequence
  F-primer
ctaatacgactcactataggaaatgttgttatagtatcccacctttttttttttaagcactgc

SEQ ID NO: 25 - DNA, 57 bp, Artificial Sequence
  R-primer
agcatattgttatactattccacccgctgcggcgtgcagtgcttaaaaaaaaaaagg

SEQ ID NO: 26 - RNA, 80 nt, Artificial Sequence
  gRNA
ggaaauguuguuauaguaucccaccuuuuuuuuuuuaagcacugcacgccgcagcggguggaauaguauaacaauaugcu

SEQ ID NO: 27 - DNA, 52 bp, Artificial Sequence
  F-primer
ctaatacgactcactataggaaatgttgttatagtatcccacctcgacgttg

SEQ ID NO: 28 - DNA, 47 bp, Artificial Sequence
  R-primer
gtgcttgaattcactggccgtcgttttacaacgtcgaggtgggatac

SEQ ID NO: 29 - DNA, 41 bp, Artificial Sequence
  F-primer
gacggccagtgaattcaagcactgcacgccgcagcgggtgg

SEQ ID NO: 30 - DNA, 39 bp, Artificial Sequence
  R-primer
agcatattgttatactattccacccgctgcggcgtgcag

SEQ ID NO: 31 - DNA, 20 bp, Artificial Sequence
  F-primer
ctaatacgactcactatagg

SEQ ID NO: 32 - DNA, 24 bp, Artificial Sequence
  R-primer
agcatattgttatactattccacc

SEQ ID NO: 33 - RNA, 100 nt, Artificial Sequence
  gRNA
ggaaauguuguuauaguaucccaccucgacguuguaaaacgacggccagugaauucaagcacugcacgccgcagcggguggaauaguauaacaauaugcu

SEQ ID NO: 34 - DNA, 77 bp, Artificial Sequence
  F-primer
ctaatacgactcactataggaaccatgccgactgatggcagcgacgttgtaaaacgacggccagtgaattcaagcac

SEQ ID NO: 35 - DNA, 62 bp, Artificial Sequence
  R-primer
ctacagtccacgccgactgatggcaggctgcggcgtgcagtgcttgaattcactggccgtcg

SEQ ID NO: 36 - RNA, 98 nt, Artificial Sequence
  gRNA
ggaaccaugccgacugauggcagcgacguuguaaaacgacggccagugaauucaagcacugcacgccgcagccugccaucagucggcguggacuguag

SEQ ID NO: 37 - DNA, 53 bp, Artificial Sequence
  F-primer
ctaatacgactcactataggcgacgttgtaaaacgacggccagtgaattcaag

SEQ ID NO: 38 - DNA, 36 bp, Artificial Sequence
  R-primer
gctgcggcgtgcagtgcttgaattcactggccgtcg

SEQ ID NO: 39 - RNA, 51 nt, Artificial Sequence
  gRNA
ggcgacguuguaaaacgacggccagugaauucaagcacugcacgccgcagc

SEQ ID NO: 40 - DNA, 40 bp, Artificial Sequence; identical to SEQ ID NO: 20
  F-primer

SEQ ID NO: 41 - DNA, 20 bp, Artificial Sequence
  R-primer
tagcgtgagaagcactgcac

SEQ ID NO: 42 - DNA, 21 bp, Artificial Sequence
  F-primer
ctaatacgactcactataggg

SEQ ID NO: 43 - RNA, 936 nt, Artificial Sequence
  modified Ruc
auggcuuccaagguguacgaccccgagcaacgcaaacgcaugaucacugggccucaguggugggcucgcugcaagcaaaugaacgugcuggacuccuucaucaacuacuaugauuccgagaggcacgccgagaacgccgugauuuuucugcaugguaacgcugccuccagcuaccuguggaggcacgucgugccucacaucgagcccguggcuagaugcaucaucccugaucugaucggaauggguaaguccggcaagagcgggaauggcucauaucgccuccuggaucacuacaaguaccucaccgcuuaguucgagcugcugaaccuuccaaagaaaaucaucuuugugggccacgacuggggggcuugucuggccuuucacuacuccuacgagcaccaagacaagaucaaggccaucguccaugcugagagugucguggacgugaucgaguccugggacgaguggccugacaucgaggaggauaucgcccugaucaagagcgaagagggcgagaaaauggugcuugagaauaacuucuucgucgagaccaugcucccaagcaagaucaugcggaaacuggagccugaggaguucgcugccuaccuggagccauucaaggagaagggcgagguuagacggccuacccucuccuggccucgcgagaucccucucguuaagggaggcaagcccgacgucguccagauuguccgcaacuacaacgccuaccuucgggccagcgacgaucugccuaagauguucaucgaguccgacccuggguucuuuuccaacgcuauugucgagggagcuaagaaguucccuaacaccgaguucgugaaggugaagggccuccacuucagccaggaggacgcuccagaugaaauggguaaguacaucaagagcuucguggagcgcgugcugaagaacgagcaguaa

SEQ ID NO: 44 - RNA, 74 nt, Artificial Sequence
  gRNA
gaagguucagcagcucgaaccaagggguggaauaguauaacaauaugcuaaauguuguuauaguaucccaccuu

SEQ ID NO: 45 - RNA, 104 nt, Artificial Sequence
  gRNA
aaauguuguuauaguaucccaccucgacguuguaaaacgacggccagugaauucgaagguucagcagcucgaaccaagggguggaauaguauaacaauaugcuu

SEQ ID NO: 46 - RNA, 304 nt, Artificial Sequence
  gRNA
gccaucagucgccggucccaagcccggauaaaaugggagggggcgggaaaccgccuaaccaugccgacugauggcaggagacggucggguccacgaaauguuguuauaguaucccaccucgacguuguaaaacgacggccagugaauucgaagguucagcagcucgaaccaagggguggaauaguauaacaauaugcucgugucgaguagagugugggcuccugccaucagucggcguggacuguagaacacugccaaugccggucccaagcccggauaaaaguggaggguacaguccacgcuu

SEQ ID NO: 47 - DNA, 37 bp, Artificial Sequence
  R-primer
ggccacgcgtcgactagtacttttttttttttttttt

SEQ ID NO: 48 - DNA, 20 bp, Artificial Sequence
  F-primer
catggcttccaaggtgtacg

SEQ ID NO: 49 - DNA, 20 bp, Artificial Sequence
  R-primer
ggccacgcgtcgactagtac

SEQ ID NO: 50 - DNA, 20 bp, Artificial Sequence
  F-primer
gcatgatcactgggcctcag

SEQ ID NO: 51 - DNA, 19 bp, Artificial Sequence
  R-primer
ccagtcgtggcccacaaag

SEQ ID NO: 52 - DNA, 19 bp, Artificial Sequence
  F-primer
gctggactccttcatcaac

SEQ ID NO: 53 - DNA, 18 bp, Artificial Sequence
  F-primer
cctcgacgttgtaaaacg

SEQ ID NO: 54 - DNA, 18 bp, Artificial Sequence
  R-primer
catggttctacagtccac

SEQ ID NO: 55 - RNA, 225 nt, Artificial Sequence
  gRNA
gggggcgggaaaccgccuaaccaugccgacugauggcaggagacggucggguccacgaaauguuguuauaguaucccaccucgacguuguaaaacgacggccagugaauucgaagguucagcagcucgaaccaagggguggaauaguauaacaauaugcucgugucgaguagagugugggcuccugccaucagucggcguggacuguagaacacugccaaugcuu

SEQ ID NO: 56 - RNA, 191 nt, Artificial Sequence
  gRNA
aaccaugccgacugauggcaggagacggucggguccacgaaauguuguuauaguaucccaccucgacguuguaaaacgacggccagugaauucgaagguucagcagcucgaaccaagggguggaauaguauaacaauaugcucgugucgaguagagugugggcuccugccaucagucggcguggacuguag

SEQ ID NO: 57 - RNA, 68 nt, Artificial Sequence
  gRNA
aagcacugcacgccgcagcggguggaauaguauaacaauaugcuaaauguuguuauaguaucccaccu

SEQ ID NO: 58 - RNA, 66 nt, Artificial Sequence
  gRNA
aagcacugcacgccgcagccugccaucagucggcguggacuguagaaccaugccgacugauggcag

SEQ ID NO: 59 - RNA, 96 nt, Artificial Sequence
  gRNA
aaccaugccgacugauggcagcgacguuguaaaacgacggccagugaauucaagcacugcacgccgcagccugccaucagucggcguggacuguag

SEQ ID NO: 60 - RNA, 212 nt, Artificial Sequence
  gRNA
gccaucagucgccggucccaagcccggauaaaaugggagggggcgggaaaccgccuaaccaugccgacugauggcagcgacguuguaaaacgacggccagugaauucaagcacugcacgccgcagccugccaucagucggcguggacuguagaacacugccaaugccggucccaagcccggauaaaaguggaggguacaguccacgcuuuuu

SEQ ID NO: 61 - RNA, 96 nt, Artificial Sequence; identical to SEQ ID NO: 59
  gRNA

SEQ ID NO: 62 - RNA, 68 nt, Artificial Sequence
  gRNA
gucaccagggugggccaggggguggaauaguauaacaauaugcuaaauguuguuauaguaucccaccu

SEQ ID NO: 63 - RNA, 66 nt, Artificial Sequence
  gRNA
gucaccagggugggccaggcugccaucagucggcguggacuguagaaccaugccgacugauggcag

SEQ ID NO: 64 - RNA, 96 nt, Artificial Sequence; identical to SEQ ID NO: 59
  gRNA

SEQ ID NO: 65 - RNA, 212 nt, Artificial Sequence
  gRNA
gccaucagucgccggucccaagcccggauaaaaugggagggggcgggaaaccgccuaaccaugccgacugauggcagcgacguuguaaaacgacggccagugaauucgucaccagggugggccaggcugccaucagucggcguggacuguagaacacugccaaugccggucccaagcccggauaaaaguggaggguacaguccacgcuuuuu

SEQ ID NO: 66 - RNA, 96 nt, Artificial Sequence
  gRNA
aaccaugccgacugauggcagcgacguuguaaaacgacggccagugaauucgucaccagggugggccaggcugccaucagucggcguggacuguag

SEQ ID NO: 67 - RNA, 73 nt, Artificial Sequence
  gRNA
gaagguucagcagcucgaaccaagggguggaauaguauaacaauaugcuaaauguuguuauaguaucccaccu

SEQ ID NO: 68 - RNA, 71 nt, Artificial Sequence
  gRNA
gaagguucagcagcucgaaccaagcugccaucagucggcguggacuguagaaccaugccgacugauggcag

SEQ ID NO: 69 - RNA, 101 nt, Artificial Sequence
  gRNA
aaccaugccgacugauggcagcgacguuguaaaacgacggccagugaauucgaagguucagcagcucgaaccaagcugccaucagucggcguggacuguag

SEQ ID NO: 70 - RNA, 217 nt, Artificial Sequence
  gRNA
gccaucagucgccggucccaagcccggauaaaaugggagggggcgggaaaccgccuaaccaugccgacugauggcagcgacguuguaaaacgacggccagugaauucgaagguucagcagcucgaaccaagcugccaucagucggcguggacuguagaacacugccaaugccggucccaagcccggauaaaaguggaggguacaguccacgcuuuuu

SEQ ID NO: 71 - RNA, 101 nt, Artificial Sequence; identical to SEQ ID NO: 69
  gRNA

SEQ ID NO: 72 - RNA, 78 nt, Artificial Sequence
  gRNA
ggaaccaugccgacugauggcaguuuuuuuuuuaagcacugcacgccgcagccugccaucagucggcguggacuguag

SEQ ID NO: 73 - RNA, 78 nt, Artificial Sequence
  gRNA
ggaaccaugccgacugauggcagaucggaauucaagcacugcacgccgcagccugccaucagucggcguggacuguag

SEQ ID NO: 74 - RNA, 76 nt, Artificial Sequence
  gRNA
ggaaccaugccgacugauggcagcggaauucaagcacugcacgccgcagccugccaucagucggcguggacuguag

SEQ ID NO: 75 - RNA, 74 nt, Artificial Sequence
  gRNA
ggaaccaugccgacugauggcaggaauucaagcacugcacgccgcagccugccaucagucggcguggacuguag

SEQ ID NO: 76 - RNA, 72 nt, Artificial Sequence
  gRNA
ggaaccaugccgacugauggcagauucaagcacugcacgccgcagccugccaucagucggcguggacuguag

SEQ ID NO: 77 - RNA, 98 nt, Artificial Sequence; identical to SEQ ID NO: 36
  gRNA

SEQ ID NO: 78 - RNA, 88 nt, Artificial Sequence
  gRNA
ggaaccaugccgacugauggcagguuguuguuguuguuguuguaagcacugcacgccgcagccugccaucagucggcguggacuguag

SEQ ID NO: 79 - RNA, 19 nt, Artificial Sequence
  target
gcuacggcgugcagugcuu